SEQUENCE LISTING (13 sequences)

SEQ ID NO: 1 - DNA, 40 bp, Artificial Sequence
  Description of Artificial Sequence Synthetic oligonucleotide
  CA40 aptamer, nucleotides 1-13 and 29-40 stem portion, nucleotides 14-28 G-quadruplex binding pocket
acgctccggacttgggtgggtgggttggggtacggtgcgt

SEQ ID NO: 2 - DNA, 40 bp, Artificial Sequence
  Description of Artificial Sequence Synthetic oligonucleotide
  CA40_1MM aptamer, nucleotides 1-13 and 29-40 stem portion, nucleotides 14-28 G-quadruplex binding pocket
acgctccggacttgggtgggtgggttggggtacggtgtgt

SEQ ID NO: 3 - DNA, 40 bp, Artificial Sequence
  Description of Artificial Sequence Synthetic oligonucleotide
  CA40_2MM aptamer, nucleotides 1-13 and 29-40 stem portion, nucleotides 14-28 G-quadruplex binding pocket
acgctctggacttgggtgggtgggttggggtacggtgtgt

SEQ ID NO: 4 - DNA, 36 bp, Artificial Sequence
  Description of Artificial Sequence Synthetic oligonucleotide
  CA36 aptamer, nucleotides 1-11 and 28-36 stem portion, nucleotides 12-27 G-quadruplex binding pocket
gctccggacttgggtgggtgggttggggtacggtgc

SEQ ID NO: 5 - DNA, 32 bp, Artificial Sequence
  Description of Artificial Sequence Synthetic oligonucleotide
  CA32 aptamer, nucleotides 1-9 and 26-32 stem portion, nucleotides 1-25 G-quadruplex binding pocket
tccggacttgggtgggtgggttggggtacggt

SEQ ID NO: 6 - DNA, 28 bp, Artificial Sequence
  Description of Artificial Sequence Synthetic oligonucleotide
  CA28 aptamer, nucleotides 1-7 and 24-28 stem portion, nucleotides 8-23 G-quadruplex binding pocket
cggacttgggtgggtgggttggggtacg

SEQ ID NO: 7 - DNA, 16 bp, Artificial Sequence
  Description of Artificial Sequence Synthetic oligonucleotide
  CA16 aptamer and sequence of the G-quadruplex binding pocket
gggtgggtgggttggg

SEQ ID NO: 8 - DNA, 10 bp, Artificial Sequence
  Description of Artificial Sequence Synthetic oligonucleotide
  linear probe sequence
attatttttt

SEQ ID NO: 9 - DNA, 5 bp, Artificial Sequence
  Description of Artificial Sequence Synthetic oligonucleotide
attat

SEQ ID NO: 10 - DNA, 15 bp, Artificial Sequence
  Description of Artificial Sequence Synthetic oligonucleotide
attattttttattta

SEQ ID NO: 11 - DNA, 30 bp, Artificial Sequence
  Description of Artificial Sequence Synthetic oligonucleotide
attattttttatttatttttattttatttt

SEQ ID NO: 12 - DNA, 40 bp, Artificial Sequence
  Description of Artificial Sequence Synthetic oligonucleotide
attattttttatttatttttattttattttattttttatt

SEQ ID NO: 13 - DNA, 60 bp, Artificial Sequence
  Description of Artificial Sequence Synthetic oligonucleotide
attattttttatttatttttattttattttattttttatttatttttattttattttatt

(19) United States
(12) Patent Application Publication
(10) Pub. No.: US 2022/0225910 A1
(43) Pub. Date: Jul. 21, 2022

(54) Modulating Electron Transfer Kinetics in E-DNA-type Sensors

(71) Applicant: The Regents of the University of California, Oakland, CA (US)

(72) Inventors: Kevin Plaxco, Santa Barbara, CA (US); Netzahualcoyotl Arroyo Curras, Baltimore, MD (US); Andrea Idili, Rome (IT)

(73) Assignee: The Regents of the University of California, Oakland, CA (US)

(21) Appl. No.: 17/617,100

(22) PCT Filed: Jun. 12, 2020
(86) PCT No.: PCT/US2020/037612

§ 371 Date: Dec. 7, 2021

Related U.S. Application Data
(60) Provisional application No. 62/860,772, filed on Jun. 12, 2019.

(51) Int. Cl. A61B 5/1473; A61B 5/1495

(52) U.S. Cl. CPC A61B 5/14735; A61B 5/1495

(57) ABSTRACT

Improved electrochemical sensors wherein the recognition element is co-deposited with a secondary, charge transfer modulating moiety, for example an oligonucleotide. The secondary moiety modulates electron transfer kinetics to enhance the frequency dependence of sensor gain, enabling the use of kinetic differential drift correction techniques and like measurements that require a target insensitive signal drifts in parallel with target-dependent output. The secondary moiety also increases the gain and signal to noise of the sensor and can be used to enable calibration-free measurement. Accurate drift-corrected in vivo sensor use with multiple measurements of analyte concentration per minute in flowing blood is demonstrated.

### CROSS-REFERENCE TO RELATED APPLICATIONS

This application is a 35 USC § 371 national stage application of PCT/US2020/037612, entitled “Modulating Electron Transfer Kinetics in E-DNA-type Sensors,” filed Jun. 12, 2020, which claims the benefit of priority to U.S. Provisional Application Ser. No. 62/860,772, entitled “Modulating Electron Transfer Kinetics in E-DNA-type Sensors,” filed Jun. 12, 2019, the contents which applications are hereby incorporated by reference.

### STATEMENT REGARDING FEDERALLY SPONSORED RESEARCH OR DEVELOPMENT

This invention was made with government support under grant number R01 EB022015 awarded by the National Institutes of Health. The Government has certain rights in the invention.

### REFERENCE TO SEQUENCE LISTING, A TABLE, OR A COMPUTER PROGRAM LISTING COMPACT DISK APPENDIX

The instant application contains a Sequence Listing which has been filed electronically in ASCII format and is hereby incorporated by reference in its entirety. Said ASCII copy, created on Jun. 12, 2020, is named UCSB029PCT_SL.txt and is 4,207 bytes in size

### BACKGROUND OF THE INVENTION

A major objective in the field of biosensing is the continuous (or at least high-frequency), real-time measurement of target analytes in complex samples and environments, such as those found in vivo. If such an ability could be achieved for a broad array of target analytes, it would open the door to countless applications in the fields of research, environmental monitoring, industry, and medicine. Many reagentless electrochemical sensors comprise a recognition element, such as an aptamer or protein, which selectively binds to a target molecule, and sensing is enabled where such binding induces a change in the kinetics of electron transfer between the recognition element and other elements of the sensor. Such sensors will be referred to herein broadly as “receptor-based sensors,” and these encompass many platforms, including DNA-based and aptamer-based electrochemical sensors (“E-DNA” and “E-AB” sensors respectively). Like most sensors, reagentless electrochemical sensors are prone to drift over time, wherein the sensor output changes independently of target analyte concentration, especially when the sensors are exposed to complex samples such as whole blood or other bodily fluids.

In some cases, sensor drift may be corrected by the use of kinetic differential measurement (KDM) technique, and like drift correction methods. KDM self-corrects for signal drift and enhances the signal to noise ratio, for example, as described in Ferguson et al., Real-Time, Aptamer-Based Tracking of Circulating Therapeutic Agents in Living Animals, Science Translational Medicine 27 Nov. 2013, Vol. 5, Issue 213, pp. 213ra165. This technique exploits the square wave frequency dependence of signaling in this class of sensors. Specifically, electron transfer is more rapid from the target-bound receptor than it is from the target-free receptor. This kinetic difference results in a binding-induced increase or decrease in current when square-wave voltammetry is performed at higher frequencies and the opposite at lower frequencies. Conveniently, these two outputs drift in concert, and thus taking their difference effectively corrects for baseline drift.

For suitable recognition elements, KDM provides a convenient way to correct for drift. However, the implementation of KDM depends on there being a strong dependence between signal behavior and the frequency of electrode interrogation. Specifically, for successful implementation of KDM, there must be at least one responsive frequency at which target binding significantly alters the signal, and at least one non-responsive or minimally responsive or negatively responsive frequency, wherein sensor output is completely or largely independent of target concentration or even decreases in response to target. That is, at the selected responsive frequency, gain should be high to impart sensitivity and/or high dynamic range and at the selected non-responsive frequency, gain should be minimal, zero, or negative, in order to enable KDM or like corrective adjustments that capture sensor signal attributable to drift. For example, for many aptamer recognition elements, target binding creates a signal-on behavior at high frequencies, and a signal-off or non-responsive output at low frequencies.

However, some recognition elements, for example some aptamers, are so constituted that their signal output is not a strong function of frequency, e.g., target binding induces a signal-on response across the standard range of SWV frequencies. For example, when an E-AB sensor using an aptamer that binds camptothecins is used as the recognition element for detection of the chemotherapeutic agent irinotecan, the sensor has substantial gain across all frequencies. There is no range of frequencies wherein gain is low, and thus there is no reference point to account for the natural drift of the sensor over time. For such frequency-insensitive recognition elements, KDM may not be implemented in standard sensor configurations. Accordingly, there is a need in for novel sensor configurations and measurement techniques that enable drift correction in receptor-based sensors that would otherwise not be amenable to these approaches.

Another issue in the use of receptor-based sensors is the need for calibration. The raw, absolute output signal produced by receptor-based sensors varies significantly from sensor to sensor due to variations in fabrication. This variability is likely due to differences in the microscopic surface area of sensor electrodes and thus the total number and packing density of redox reporters that are exchanging electrons. Likewise, non-specific binding by non-target species present in the sample will often cause variability in sensor output. In controlled contexts, these problems can be circumvented by a calibration step wherein output is measured when the sensor is exposed to a sample of known target concentration. Such calibration may be sufficient for many applications, for example, continuous monitoring of an exogenously applied target or in benchtop applications wherein blank or standard samples are readily available. However, the critical calibration process cannot be applied in many settings, for example in the case of in vivo monitoring. Even for ex vivo applications, such as point-of-care applications, the need for calibration increases the complexity, time, and expense of the method and in many cases the calibration steps are beyond the practical or economic limits of clinical or bedside applications. Thus, there is a need in the art for calibration-free measurement methods that can accurately quantify the concentration of a given molecular analyte irrespective of sensor-to-sensor variation or sensor drift.

### SUMMARY OF THE INVENTION

The inventors of the present disclosure have advantageously discovered methods of modulating electron transfer kinetics in receptor-based sensors by use of a secondary moiety co-deposited with the recognition element. The secondary moiety does not respond to the presence of the target, but does modulate the frequency dependence of electron transfer by the sensor system. It may do so by transferring electrons itself. Or it may do so by modulating the electron transfer behavior of the target-binding recognition element. By the inclusion of these molecules in combination with the recognition element, the kinetics of electron transfer between the recognition element and the electrode may modulated with beneficial effects. First, the gain of the sensor may be increased. Secondly, the frequency-dependence of the sensor's gain may be enhanced.

In a first aspect, the scope of the invention encompasses novel sensor designs, wherein the recognition elements of receptor-binding sensors are co-deposited with a secondary charge transfer-modulating moiety on the sensing electrode. In some embodiments, the sensor is a DNA-type sensor, such as an aptamer-based sensor. In some embodiments, the secondary charge transfer-modulating moiety is a redox-reporter labeled oligonucleotide, such as a short oligonucleotide of 2 to 20 bases. In some embodiments, the secondary charge transfer-modulating moiety is an oligonucleotide, such as a short oligonucleotide of 2 to 20 bases, lacking a redox-reporter.

In another aspect, the scope of the invention encompasses the use of the novel sensors of the invention for drift correction. The secondary charge transfer-modulating moiety provides a reference signal that is insensitive to target concentration, and which drifts in parallel with the measurement signal generated by the recognition elements. Thus, drift effects may be quantified and subtracted from measured signal to provide drift-corrected measurements.

In another aspect, the gain of an electrochemical sensor is enhanced by use of a sensing element comprising a recognition element co-deposited with a secondary charge transfer-modulating moiety. Enhanced gain enables sensor operation with challenging sample types, such as whole blood, for example flowing whole blood in vivo, wherein high signal-to-noise ratio is necessary for resolving target concentration.

In another aspect, the novel sensors of the invention may be utilized in calibration-free, operation. By the co-deposited secondary charge transfer-modulating moiety, stronger frequency dependence of the gain may be generated. Using the frequency dependence of gain, normalized sensor outputs may be obtained, accounting for sensor-to-sensor variation that affects absolute outputs. The ability to utilize the electrochemical sensors of the invention in a calibration-free operation enables extended use in vivo or in other contexts wherein calibration is cumbersome or not practical.

In another aspect, the scope of the invention encompasses novel camptothecin binding aptamers. The novel aptamers are impart higher gain than prior art camptothecin aptamers when used in electrochemical sensing platforms.

### BRIEF DESCRIPTION OF THE DRAWINGS

FIGS. 1A, 1B, 1C, and 1D. FIG. 1A is a diagram depicting an exemplary prior art electrochemical aptamer-based (E-AB) sensor. The diagram depicts an unbound aptamer 103 modified a redox reporter 107, covalently attached to a gold electrode 101 via an alkane-thiol self-assembled monolayer 102. In the absence of its specific target, the aptamer is partially or entirely unfolded (103). When a target molecule 104 binds to the aptamer 105, a conformational change alters the proximity of the redox reporter 108 and improves the efficiency of electron transfer signal (star) to the electrode observed upon voltammetric (such as square-wave) interrogation. FIG. 1B: In the completed sensor a 75 mm gold-wire working electrode is bundled with same-diameter platinum counter electrode and a silver/silver-chloride reference electrode, creating a device small enough and flexible enough to be emplaced via a 22-gauge guide catheter in one of the external jugular veins of a live rat. FIG. 1C depicts sensor placement in rat 101, with sensor bundle 102 external to the animal and the sensing electrode 103 placed in the jugular vein 104. FIG. 1D depicts the different signal outputs observed in the absence and presence of target.

FIGS. 2A, 2B, 2C, and 2D. FIG. 1A: The parent aptamer is predicted to fold into a G-quadruplex, which is thought to be the target-binding site, flanked by a 12-base-pair stem. FIG. 2B: Exploiting the intrinsic fluorescence of irinotecan, when free in solution, the aptamer exhibits a dissociation constant of 475 nM. FIG. 2C: When the aptamer is redox-reporter-modified and anchored to the sensor's interrogating electrode, however, its affinity and signal gain are reduced significantly, particularly when deployed in undiluted whole blood, these binding curves employed a square-wave frequency of 120 Hz. FIG. 1D: The E-AB sensor nevertheless rapidly responds to when challenged (here in buffer) with irinotecan. These binding curves employed a square-wave frequency of 500 Hz and a repetition rate of 0.2 Hz.

FIGS. 3A, 3B, and 3C. FIG. 3A: The parent aptamer was re-engineered to produce higher-gain E-AB signaling by destabilizing the aptamer's stem-loop (thus increasing the population of unfolded molecules poised to respond to target) via either introduction of one (CA40_1MM) or two (CA40_2MM) mismatches or via truncation (CA36, CA32, CA28, CA16) of the stem. FIG. 3B: When challenged in a simple buffered solution all of the re-engineered variants exhibited higher gain than that of the parent aptamer, with the most destabilized (CA40_2MM, CA32, CA28, CA16) producing the greatest signal gain. FIG. 3C: When tested in whole blood their gain and affinity are reduced, but the best performing nevertheless still support high-gain E-AB sensing.

FIGS. 4A, 4B, and 4C. FIG. 4A is a diagram depicting a sensor of the invention. The sensor comprises a gold electrode 101 functionalized with a SAM layer 102 by which a target-binding aptamer 103, 107 modified with a redox reporter is co-deposited with a secondary moiety, in this case a redox reporter-modified oligonucleotide strand 105. An unbound aptamer 103 is shown. When the aptamer 107 binds target molecule 108, it undergoes a conformational change, wherein proximity of the redox reporter 109 to the electrode 101 changes the electron transfer kinetics and resulting signal. FIG. 4B: The signal gain (relative signal change between no target and saturating target—i.e., 100 mM) of the original E-AB sensor (100:0 curve) is a relatively minor function of square-wave frequency. Upon co-deposition with increasing amounts of the linear-strand (to a maximum ratio of 50:50) increasingly strong frequency dependence is observed, albeit with a corresponding reduction in the maximum gain. FIG. 4C depicts a sensor fabricated using a 50:50 mixture of the two strands and employing KDM drift correction (the difference in the relative signals seen at 10 and 120 Hz) responds to target over the clinically-relevant range (0.5 mM to 15 mM) in both buffer and in undiluted whole blood.

FIGS. 5A and 5B. FIG. 5A: Sensor output over time in response to an intravenous injection of irinotecan (60 mg kg¹) from time 20 to ˜55 minutes. FIG. 5B: Closeup of excretion phase, from ˜55 to 86 minutes. The higher peak concentrations reached in this experiment lead to longer measurement runs, in turn improving the precision of the estimates of the relevant pharmacokinetic parameters.

FIGS. 6A, 6B, and 6C. FIG. 6A depicts signals collected at high (120 Hz) and low (10 Hz) frequencies by a sensor of the invention. Both signals drift significantly, but they drift in concert. FIG. 6B: taking the difference (KDM) between the normalized high (120 Hz) and low (10 Hz) frequency signals produces a stable baseline. FIG. 6C: control injections of either a saline “blank” or a second chemotherapeutic (5-fluorouracil, which is often co-administered with irinotecan) do not produce any measurable sensor response.

FIG. 7. FIG. 7 depicts measured irinotecan levels following multiple intravenous injections in a live rat. The black lines represent the fit of each injection dataset to a two-compartment pharmacokinetic model.

FIGS. 8A and 8B. FIGS. 8A and 8B depict E-AB sensor output collected in vitro in either buffer (FIG. 8A) or flowing whole blood (FIG. 8B). In buffer a K_{D}=16.0±1.3 μM is observed for the KDM signal. In whole blood K_{D} rises to 191.2±23.4 μM. Error bars correspond to the standard deviations observed across three independently fabricated and tested sensors.

FIGS. 9A, 9B, 9C, 9D, 9E, and 9F. FIGS. 9A, 9B, 9C, 9D, 9E, and 9F depicts frequency effect on signal gain for 100% C28 variant aptamer (SEQ ID NO: 6) and mixtures of C28 aptamer and oligonucleotide secondary moiety functionalized with —SH at the 5′ end for attachment to the SAM functionalized gold electrode surface and functionalized at the 3′ end with methylene blue. Signal depicted is average of 3 sensors, irinotecan at 100 μM, dissolved in DMSO, in PBS at room temperature; E_{increment}=0.001 V; Amplitude=0.05 V. FIGS. 9A, 9C, 9D, 9E, and 9F depict signals from pure CA28 (“100:0”) and 50-50% mixtures of C28 and oligonucleotide (“50:50”). FIG. 9A: 5 nt oligonucleotide secondary moiety, SEQ ID NO: 9. FIG. 9B depicts pure CA28 (“100:0”) and various mixtures of C28 and 10 nt oligonucleotide secondary moiety, SEQ ID NO: 8 (50:50-50% CA29 and 50% secondary moiety. 40:60-40% CA28 and 60% secondary moiety, 20:80-20% CA28 and 80% secondary moiety). FIG. 9C: 15 nt oligonucleotide secondary moiety, SEQ ID NO: 10. FIG. 9D: 30 nt oligonucleotide secondary moiety, SEQ ID NO: 11. FIG. 9E: 40 nt oligonucleotide secondary moiety, SEQ ID NO: 12. FIG. 9F: 60 nt oligonucleotide secondary moiety, SEQ ID NO: 13

### DETAILED DESCRIPTION OF THE INVENTION

The scope of the invention encompasses novel electrochemical sensor designs and methods of use thereof. The invention is directed particularly to electrochemical sensor that can be collectively referred to as “receptor-based electrochemical sensors.” A receptor-based electrochemical sensor comprises a recognition element functionalized with a redox reporter, wherein the recognition element selectively binds to a target molecule. The redox reporter-functionalized recognition element is bound to or otherwise associated with an electrode. When energized by application of a voltage and/or current pulse, the redox reporter exchanges electrons with the electrode, generating a measurable signal. Binding of the target molecule to the recognition element modulates the kinetics of this electron transfer and the resulting change in signal is correlated with target concentration.

The electrochemical sensors of the invention comprise a secondary charge transfer-modulating moiety that modulates the signal from the recognition element and provides various benefits. First, the secondary moiety can improve the gain of the sensor. Secondly, the secondary moiety can impart or enhance the frequency dependence of the sensor's output signal, enabling the use of drift correction methods and calibration-free operation of the sensor. The various elements and embodiments of the invention are described next.

Electrochemical sensors. The improved sensor designs of the invention may be applied to any electrochemical sensor. In a primary embodiment, the sensors of the invention will be E-DNA type sensors, employing a target-binding polynucleotide such as an aptamer as the recognition element. Any E-DNA sensor design or configuration known in the art may be used. In some implementations, the E-DNA sensor is an E-AB sensor, wherein the recognition element comprises an aptamer, as known in the art. The polynucleotide recognition element may comprise DNA, RNA, or an non-natural nucleic acids, as well as hybrids of the foregoing.

In an E-DNA sensor, one or more selected portions of the working electrode are functionalized with a polynucleotide. The polynucleotide may be conjugated to or otherwise associated with the electrode surface by any appropriate chemistry, for example by covalent bonding, chemisorption, or adsorption. Alkane thiol monolayers may be used to conjugate polynucleotides to the electrode surface, being particularly suitable for gold electrode surfaces.

The scope of the invention is not limited to E-AB sensors. The scope of the invention further encompasses any electrochemical sensor wherein target binding to recognition elements creates measurable changes in electron transfer rates detectable by the sensing element. In various embodiments, the recognition element is other than a nucleic acid, for example sensors using proteins (e.g. antibodies or fragments thereof), chemical species, and other molecules as the recognition element are also within the scope of the invention.

In one implementation, sensors that employ non-mediated electrochemical sensing may be used, including the use of direct electron transfer and redox equilibrium as a way to generate a signal. These biochemical sensors include, for example, sensors that detect the consumption or generation of species belonging to redox couples. Other sensors may employ a mediated electrochemical analysis, i.e. using a redox species mediator for electron transfer and establishing redox equilibrium. Examples of mediated and non-mediated sensors can be found in Sander et al. 2015, A Review of Nonmediated and Mediated Approaches. Environ. Sci. Technol. 49:5862-5878.

Additional sensor types include chemically modified electrodes, immunosensors, oligopeptide-based sensors, and enzymatic sensors.

Another sensor type that may be used is a sensor based on electron transfer changes due to target binding-induced displacement of ligands, for example, hexamethylphosphoramide with samarium (II) iodide as described in Prasad, 2004, The Role of Ligand Displacement in Sm(II)-IMPA-Based Reductions. J Am Chem Soc. 2004; 126(22):6891-4.

Another exemplary sensor type is based on changes in a redox reporter's reorganizational energy, for example, ferrocenoyl-peptides as described in Plumb, 2003, Interaction of a Ferrocenoyl-Modified Peptide with Papain: Toward Protein-Sensitive Electrochemical Probes. Bioconj Chem. 2003; 14(3):601-6. doi: 10.1021/bc0256446; or trinuclear ruthenium clusters, for example, as described in Feld 2012, Trinuclear Ruthenium Clusters as Bivalent Electrochemical Probes for Ligand-Receptor Binding Interactions. Langmuir. 2012; 28(1):939-49. doi: 10.1021/la202882k.

Another sensor type that may be used is a sensor based on sterically induced changes in the efficiency with which a scaffold-attached redox reporter approaches an underlying electrode surface, for example, duplex DNA, quadruplex DNA, and DNA nanoswitches as described in Ge 2010, A Robust Electronic Switch Made of Immobilized Duplex/Quadruplex DNA. Angew Chem Int Ed. 2010; 49(51):9965-. doi: 10.1002/anie.201004946, or DNA-containing a small molecule recognition element as described in Cash 2009, An Electrochemical Sensor for the Detection of Protein-Small Molecule Interactions Directly in Serum and Other Complex Matrices. J Am Chem Soc. 2009; 131(20):6955-7. doi: 10.1021/ja9011595.

In some implementations, the recognition element of the sensor comprises a polypeptide. For example, in one embodiment, the polypeptide may comprise a receptor, or ligand-biding domain thereof, for which the target species is a ligand. In one embodiment, the polypeptide is an antibody or antigen-binding fragment thereof, wherein the target species is the antigen.

In one embodiment, the electrochemical sensor is an E-AB sensor comprising a dual-strand sensor, wherein the redox species is present on a separate strand, a portion of which is complementary to or otherwise capable of reversibly binding to a portion of the aptamer. In the presence of the target species, the redox species' strand is liberated from the aptamer, allowing the target species to bind to the aptamer and the redox species to come into contact or proximity to the electrode.

In some implementations, the electrochemical sensor is a signal-on type sensor, such that target binding enhances the signal, and in other implementations the electrochemical sensor may comprise a signal-off configuration, wherein target binding reduces or eliminates the output signal.

Redox Reporters. In a primary implementation, each recognition element of the electrochemical sensor is functionalized with one or more redox reporters. Binding of the target species causes the recognition element to change its configuration, such that the position of (or the accessibility to the electrode of) the one or more redox reporters is detectably altered. Likewise, in many implementations of the invention, the secondary charge-modulating moiety is modified with one or more redox reporters. The redox reporters of the recognition element and/or secondary moiety may comprise any composition of matter that interacts with the electrode such that a change in its accessibility to or proximity to the electrode causes a change in the electron transfer kinetics. Exemplary redox species include methylene blue, ferrocene, viologen, anthraquinone or any other quinones, ethidium bromide, daunomycin, organo-metallic redox labels, for example porphyrin complexes or crown ether cycles or linear ethers, ruthenium, bis-pyridine, tris-pyridine, bis-imidizole, ethylenetetracetic acid-metal complexes, cytochrome c, plastocyanin, and cytochrome c′.

Sensor Components. The electrochemical sensor may comprise one or more working electrodes, to which a plurality of recognition elements are bound. Exemplary recognition element densities may be in the range of 1×10¹⁰ to 1×10¹³ molecules/cm². The working electrode may comprise any suitable electrode material for electrochemical sensing, including, for example: any metallic surface that forms a bond with thiols or amines; gold; any gold-coated metal, (such as titanium, tungsten, platinum, carbon, aluminum, copper, etc); bare palladium electrodes, carbon electrodes, etc.

The working electrode may be configured in any desired shape or size. For example, paddle-shaped electrodes, rectangular electrodes, wire electrodes, electrode arrays, screen-printed electrodes, and other configurations may be used. For in vivo measurements, a thin wire configuration is advantageous, as the low profile wire may be inserted into veins, arteries, tissue or organs and will not impede blood flow in blood vessels or cause substantial damage in tissues. For example, a wire having a diameter of 1-500 μm, for example, 50 μm, 75 μm, or 100 μm, may be used.

The electrochemical sensing systems of the invention further comprise an auxiliary or counter electrode, for example, a platinum auxiliary electrode. The electrochemical sensing element may be used with a reference electrode, for example an Ag/AgCl electrode, or other reference electrode known in the art. The electrochemical sensor of the invention may be configured in a two-electrode or a three-electrode system, appropriately configured for performing chronoamperometric measurements. The electrode-containing cell system may comprise a mixing chamber or other vessel wherein the electrodes are present and are in contact with the sample.

The sensor and electrode system may comprise an assembly for obtaining faradic current measurements when deployed in the sample or exposed to the sample. The assembly may comprise a housing. For example, for placement in the body of a living organism, the housing may comprise a needle, catheter, or other implantable structure. For ex vivo applications, the housing may comprise a well, microfluidic vessel, or other structure, such as found in a lab-on-chip device.

The sensing elements of the invention will be in functional connection with appropriate components for performing electron transfer measurements. The measurement components may comprise two or more devices in electrical and/or network connection with one another, or may comprise a single integrated device. A first component for performing measurements comprises a device or combination of devices that can deliver excitation voltage pulses of a desired magnitude, frequency, and waveform to the sensing element. Measurement components may include potientiostats or other voltage sources and voltage controllers for imposing voltage steps on the working electrode.

A second component for performing measurements comprises a device or combination of devices that can acquire time-resolved current outputs from the sensing element. These components will comprise circuitry for reading sensor outputs and storing such outputs or routing the outputs to other devices, including components as analog-to-digital converters, amplifiers, and digital storage media.

Target Species. The sensors of the invention are directed to the detection of a target species. The target species may comprise any inorganic or organic molecule, for example: a small molecule drug, a metabolite, a hormone, a peptide, a protein, a carbohydrate, a nucleic acid, a lipid, a hormone, metabolite, growth factor, neurotransmitter, or a nutrient. The target may comprise a pollutant or contaminant. The target may comprise a toxin. The target may comprise a pathogen-induced or pathogen-derived factor, or a virus or cell. In some embodiments, the target species comprises a drug having significant side effects, such as a chemotherapeutic drug, or a drug having a narrow therapeutic index, wherein accurate measurement of blood level is critical to ensure safe dosing or minimal side effects.

Secondary Moiety. The sensing elements of the invention comprise a charge transfer-modulating moiety, referred to herein as a secondary moiety, that is a non-target binding molecule that is co-deposited or otherwise present on the sensing element, In one embodiment this secondary moiety includes a redox reporter that (1) transfers electrons more rapidly or more slowly than the recognition element does and (2) does not respond to the presence of the target.

In a second embodiment the secondary moiety does not contain its own redox reporter. Instead it modulates the electron transfer rate of the redox moiety on the recognition element by interacting with the reporter or the recognition element. Such interactions can be driven by sterics, or electrostatics, or more specific interactions such as hydrogen bonding or hydrophobic interactions.

For ease of reference, the charge transfer modulating moiety may be referred to herein as “a secondary moiety.” In a primary embodiment, this will be a single species of molecule wherein a plurality of such molecule is mixed with a plurality of recognition elements on the electrode surface. However, it will be understood that the term “secondary moiety” encompasses one or more different types of molecules, for example, in some implementations, comprising a mixture of two or more different types of molecules, i.e., a heterogenous mixture, in any proportions.

One mechanism by which this modulating secondary moiety acts is by interfering with the transfer of electrons between redox reporters on the recognition element and the working electrode. This interference generally slows the rate of electron transfer between redox reporters on the recognition element and the electrode, wherein the electron transfer rates for the recognition element's bound and unbound states respond differentially to the interference created by the secondary moiety. Thus, the use of the modulating moiety can alter the frequency dependence of the target-induced currents generated during SWV or other excitation sequences. It can also improve the separation in transfer rates between the bound and unbound states of the recognition element, improving sensor gain.

The secondary moiety may be any species that can be co-deposited with recognition elements, including biomolecules such as oligonucleotides and proteins, or other compositions including polymeric organic materials or other chemical entities. In a first implementation, the secondary moiety comprises an oligonucleotide. Advantageously, in the case of E-AB and other E-DNA-type sensors, the use of oligonucleotides as modulating moieties enables co-deposition of aptamers and the secondary moieties in a single step with a single attachment chemistry (e.g., thiolated 5′ DNA ends for attachment to gold or other surfaces). In a primary embodiment, the oligonucleotides comprise DNA, although the scope of the invention encompasses other nucleic acids, including non-natural nucleic acid analogs.

The oligonucleotides may comprise any length, for example, two, three, four, five, six, seven, eight, nine, ten, eleven, twelve, thirteen, fourteen, fifteen, sixteen, seventeen, eighteen, nineteen, twenty, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 50-55, 55-60, 60-65, 65-70, 70-75, 75-80, 80-85, 85-90, 90-95-95-100, 100-110, 110-120, 120-130, 130-140, 140-150, 150-160, 160-170, 170-180, 180-190, 190-200, or more than 200 nucleotides. In general, the longer the oligonucleotide, the greater the interference effect will be on charge transfer rates between redox-modified recognition elements and the working electrode. In some embodiments, the oligonucleotide comprises 5-10, 5-20, 10-15, 10-20, or 10-50 nucleotides.

In one embodiment, the oligonucleotide comprises a sequence of thymines and adenines. In one embodiment, the secondary moiety is an oligonucleotide comprised solely of the base thymine. In one embodiment, the, the secondary moiety is an oligonucleotide comprised solely of adenines. In various embodiments, the nucleotide comprises at least 10%, at least 20%, at least 30%, at least 40%, at least 50%, at least 60%, at least 70%, at least 80%, at least 90% or at least 95% thymine. In various embodiments, the nucleotide comprises at least 10%, at least 20%, at least 30%, at least 40%, at least 50%, at least 60%, at least 70%, at least 80%, at least 90% or at least 95% adenines.

In one embodiment, the oligonucleotide comprises an oligonucleotide selected from the group consisting of SEQ ID NO: 8, SEQ ID NO: 9, SEQ ID NO: 10, SEQ ID NO: 11, SEQ ID NO: 12 SEQ ID NO: 13, or an oligonucleotide having at least 80%, at least 85%, at least 90%, at least 95%, or at least 99% sequence identity to an oligonucleotide sequence selected from the group consisting of SEQ ID NO: 8, SEQ ID NO: 9, SEQ ID NO: 10, SEQ ID NO: 11, SEQ ID NO: 12 and SEQ ID NO: 13 In various embodiments, the oligonucleotide is a subsequence of the foregoing.

In alternative embodiments, the secondary moiety comprises a polypeptide. For example, a the secondary moiety may comprise a polypeptide of 2 to 1,000 amino acids, for example, a polypeptide of 2-5, 5-10, 10-15, 15-20, 20-25, or 30 or more amino acids. Exemplary polypeptides include flexible, intrinsically disordered sequences, for example, chains of threonine, serine, proline, glycine, aspartic acid, lysine, glutamine, asparagine, or alanine, or mixtures of the foregoing. This can also include intrinsically disordered, naturally occurring proteins. This can also include folded proteins.

In alternative embodiments, the secondary moiety comprises an organic composition. In one embodiment, the organic composition comprise a polymer, such as a polyglycol, polyacid, polyalcohol, polyether, polyester, polyvinyl, or polysaccharide.

In a primary embodiment, the secondary moiety is modified with one or more redox reporters. In one embodiment, the secondary moiety is modified a single redox reporter. For example, in the case of secondary moieties comprising oligonucleotides, the redox reporter may comprise a 3′ attached moiety, for example methylene blue. The redox reporter of the secondary moiety will generally be the same chemical entity as the recognition element is modified with, however in alternative implementations, the secondary moiety may comprise a different redox reporter than that of the recognition element.

In an alternative implementation, the secondary moiety comprises no redox label. In this implementation, the electron transfer-modulating properties of the secondary moiety are entirely derived from the effects of the moiety on the charge transfer kinetics of the redox-reporter-modified recognition elements.

The absolute abundance of the charge transfer-modulating moiety may be any that is sufficient for effective signaling properties, for example, at densities of 1×10¹⁰ to 1×10¹³ molecules/cm². The relative proportion of the modulating moiety to the recognition element on the sensor will affect the degree of signal property modulation. The percentage of secondary moiety (i.e., the proportion of secondary moieties present to total recognition elements plus secondary moieties present) may vary, for example, the modulating moiety may be present at about 5%, about 10%, about 20%, about 30%, about 35%, about 40%, about 45%, about 50%, about 55%, about 60%, about 70%, about 80%, or about 90%. In one implementation, the percentage of modulating moiety is between 40 and 60%. In one implementation, the percentage of modulating moiety is about 50%. These ratios can be controlled by controlling the concentration of the two species during fabrication, or by varying their deposition time.

Methods of Use. The scope of the invention encompasses methods of using the novel sensors of the invention. General electrochemical sensor operation will be described below, followed by a description of particular methods of use wherein the novel sensing elements of the invention may be employed.

Pulsed Voltammetry. In a primary embodiment, electrochemical sensors may be utilized in a pulsed voltammetric method. A pulsed voltammetric method is an electrochemical measurement technique wherein the functionalized electrode of the sensor is subjected to a series of potentials applied in a pulsed waveform, and wherein one or more measurements of system current are assessed in each cycle. In the practice of the invention, the series of potential pulses will comprise one or more suitable potential pulses, a suitable potential pulse being an applied potential at a value that generates a measurable current output from the redox reporter species utilized in the system. Generally, the series of potential pulses will comprise pulses at, near, or encompassing (i.e. in a sweep voltage method) the redox potential(s) of the one or more redox reporter species of the sensing element, i.e. being of a voltage capable of exciting and inducing a faradaic current flow between redox reporters of the sensor's recognition elements and secondary moieties and the electrode. For example, electrical excitation of the redox reporters may induce a temporary flow of current between the redox reporters and an electrode substrate (or between the electrode substrate and the redox reporters, depending on the configuration of the system). Upon stepping the voltage of a working electrode, such as the substrate of an E-AB sensor, the working electrode becomes either a stronger reducing agent (in the case of stepping to a more negative potential) or a stronger oxidizing agent (in the case of stepping to a more positive potential). Within appropriate ranges (e.g., near or above the redox potential of the redox reporter), this step in voltage will induce a faradaic current flow between redox reporters of the sensing element and the electrode substrate. As current flows, the pool of electrons mobilized by the excitation becomes depleted, and the current will decay at rates and amplitudes dependent upon the binding status of the recognition elements of the sensor, wherein target binding induces faster or slower transfer of current, for example, by changing the proximity of the redox reporter to the working electrode. Accordingly, the ratio of target-bound recognition elements to recognition elements not bound by the target, which ratio is proportional to the concentration of the target species in the sample, will determine the observed current decay rates and amplitudes for the sensor as a whole.

As used herein, “current” will refer to the flow of electrons measured by a sensor that has been deployed in a sample. For example, current may comprise flow of electrons from redox reporters to an electrode, or may comprise the flow of electrons from an electrode to redox reporters.

In a primary embodiment, the pulsed voltammetric method comprises square wave voltammetry (SWV), as known in the art. In SWV, a square voltage waveform is applied to the working electrode and faradic current in the cell is measured. The current may be sampled twice during each square wave cycle, once at the end of the forward pulse, and again at the end of the reverse pulse. The technique discriminates against charging current by delaying the current measurement to the end of the pulse. The difference in current between the two measurements is plotted against the applied potential.

The scope of the invention further encompasses other pulsed voltammetric techniques wherein a target concentration-independent or less-concentration-dependent signal is generated at a specific frequency or frequencies. Exemplary voltammetric methods include cyclic voltammetry, differential pulse voltammetry, alternating current voltammetry, potentiometry or amperometry. Appropriate excitation waveforms may be selected, as known in the art. In addition to square waves, other waveforms may be used such as linear scans, triangular waves, differential pulses, and stepped waveforms.

Excitation pulse frequency may be selected for effective signal generation. For example, voltage steps in the range of +/−0.1 V to 0.5 V may be utilized, at repetition rates of 1 to 10,000 Hz, for example, frequencies of about 5 Hz, 10 Hz, 20 Hz, 30 Hz, 40 hz, 50 Hz, 60 Hz, 70 Hz, 80 Hz, 90 hz, 100 Hz, 110 Hz, 120 Hz, 130 Hz, 140 Hz, 150 Hz, 160 Hz, 170 Hz, 180 Hz, 190 Hz, 200 Hz, 250 Hz, 300 Hz, 350 Hz, 400 Hz, 450 Hz, 500 Hz, 600 Hz, 700 Hz, 800 Hz, 900 Hz, 1000 Hz, 1500 Hz, 2000 Hz, 3000 Hz, 4000, Hz, 5000 Hz, and 10,000 Hz, and at any intermediate value between 1 and 10,000 Hz.

Acquisition of time-resolved current measurements at time scales of microseconds to milliseconds will typically be performed. Typical current transients have a duration in the range of 10-100 ms, and may be resolved by sampling at selected time intervals, for example, intervals of about 1 ρs, 2 ρs, 3 ρs, 5 ρs, or 10 ρs, 50 ρs, 60 ρs, 70 ρs, 80 ρs, 90 ρs, 100 ρs, 200 ρs, 300 ρs 10, 400 ρs, 500 ρs, 600 ρs, 700 ρs, 800 ρs, 900 ρs, 1 ms, 5 ms, 10 ms, 20 ms, 30 ms, 40 ms, 50 ms, 60 ms, 70 ms, 80 ms, 90 ms or 100 ms or greater.

In some sensor systems, the kinetics of interconversion between the bound and unbound states of the recognition element are faster than the electron transfer events measured by the sensor. Thus, the observed current transients reflect a population-weighted average of the bound and unbound states.

Sensor deployment and Operating Conditions. Sensors are utilized under selected operating conditions, which encompass various aspects of the detection process. Operating conditions may encompass any combination of factors that affect the operation and output of the sensor.

In a first aspect, the operating conditions encompass the sample type to be analyzed. The target species of the invention are assessed in a sample. The sample will comprise a liquid. The sample may comprise whole blood, serum, saliva, urine, sweat, interstitial fluid, spinal fluid, cerebral fluid, tissue exudates, macerated tissue samples, cell solutions, intracellular compartments, water, wash water, wastewater, groundwater, food, beverages, or other biological and environmental samples. In some embodiments, the sample is derived from a subject, for example a human patient or a non-human animal such as a veterinary subject or test animal. In one embodiment, the sample comprises flowing whole blood, i.e., blood sampled by a sensing system comprising a sensor implanted in the living body (e.g., in the circulatory system) of a subject.

In one embodiment, the sample is processed prior to measurement. Examples of processing include filtering, dilution, buffering, centrifugation, and the application of other materials or processes to the sample prior to analysis. In some embodiments, the sample is not processed prior to performing measurements, for example, the sample being undiluted, unfiltered, or unconcentrated, e.g. whole blood.

In a second aspect, the operating conditions encompass the assay conditions. The general assay conditions will refer to reaction conditions for the assay, such as sample volume, temperature, pH, etc.

The calibration-free and drift-free methods of the invention are especially amenable to in vivo measurements. In one embodiment, the electrochemical sensor is configured for deployment of the sensing element in vivo. In one embodiment, the sensing element comprises a micro wire. In various implementations, the sensing element or housing of a sensing system may be inserted, implanted, or otherwise placed within the body of a living organism. The sensor element of a sensing system may be implanted in the circulatory system, subcutaneously, intraperitoneally, within an organ, or in other body compartments, wherein the sensing element is exposed to in vivo fluids, e.g., interstitial fluid, blood, for example, flowing whole blood. The implanted sensing system of the invention may comprise an implanted sensing element in connection with components external to the body (e.g. by leads, wires, or wireless communication means) wherein the external components perform pulse generation, data acquisition or processing. Alternatively, the one or more ancillary components to the sensing element, or even the entire sensing system of the invention, may be implanted in the body, with communication (by, for example, leads, wires, or wireless communication devices) to external devices for data collection.

In one embodiment, methods of the invention are performed in a feedback controlled dosing system, as known in the art, wherein a drug is administered to maintain blood concentrations within a therapeutic index or safety index. For example, in such methods and systems, the methods of the invention are applied using an electrochemical sensing element implanted in a subject to measure the concentration of a target species, wherein the target species is a drug, metabolite of a drug, or biomarker indicating that the drug should be administered. When the detected levels of the target species indicate that the subject is in need of administration of a drug or other agent, an implanted pump or other agent delivery means administers the drug or agent metered at a dosage to maintain the concentration of drug or agent within a desired range.

In other contexts, the sensors of the invention are utilized for long-term and/or continuous monitoring of environmental or industrial sites, for example, in rivers, seas, water treatment plants, industrial facilities, food processing facilities, etc.

The sensors of the invention may also be used in ex-vivo and diagnostic applications. In one embodiment, the method of the invention comprises the steps of withdrawing a sample from a living organism, and measuring the concentration of the target species in the sample by calibration-free measurement. In one embodiment, the sensors of the invention are employed in point-of-care testing systems. For example, in one embodiment, the sample is a blood sample, for example, a self-withdrawn pin-prick or finger-prick blood sample, or a urine, sweat, or saliva sample. In such embodiments, the electrochemical sensor may be deployed in a housing such as a well, slide, lab-on-chip, microfluidic chamber, or other device.

Drift Correction. In one aspect, the scope of the invention encompasses a method of measuring the concentration of a target species in a sample by the use of the novel electrochemical sensors wherein drift correction is utilized to account for signal drift. The secondary moiety of the electrochemical sensor provides a means of implementing drift correction by imparting or enhancing the frequency-dependence of sensor output.

In various implementations, the scope of the invention encompasses a method of drift-corrected measurement of the concentration of a target species in a sample by the use of an electrochemical sensor, the method comprising the steps of:
deploying the sensing element of the electrochemical sensor such that it is exposed to a sample;
wherein the sensing element comprises an electrode; wherein the electrode is functionalized with a plurality of a recognition element, wherein the recognition element is capable of selectively binding a target species and wherein the recognition element is functionalized with one or more redox reporters; and wherein the electrode is also functionalized with a plurality of a charge transfer-modulating moiety, wherein the charge-transfer modulating moiety does not substantially bind to the target species;
applying a series of excitation pulses to the sensing element at a selected non-responsive frequency to generate a reference signal, wherein the non-responsive frequency is a frequency at which the signal not measurably affected by the presence of target, is minimally responsive to the presence of target, or is negatively responsive to the presence of target;
applying a series of excitation pulses to the electrochemical sensor at a selected responsive frequency to generate a measurement signal, wherein the responsive frequency is a frequency at which the signal is dependent upon the concentration of target in the sample;
subtracting the reference signal from the measurement signal to determine a drift-corrected signal value; and applying a mathematical relationship between drift-corrected signal value and target concentration to the measured drift-corrected signal to determine the concentration of target in the sample.

The first non-responsive frequency and second responsive frequency can be determined by one of skill in the art by routine experimentation for a particular sensor, or a particular sensor class (i.e., sensors of the same design and composition, sensors manufactured in a particular lot, or other groupings of sensors expected to have like behavior), under a selected set of operating conditions, wherein sensor output is measured across a range of frequencies and a range of target concentrations to determine those frequencies wherein signal output is independent of target concentration and those where output is responsive to target concentration. In some implementations, the non-responsive frequency is a relatively lower frequency and the responsive frequency is a relatively higher frequency. In various embodiments, the non-responsive frequency may comprise a frequency at which the signal does not measurably respond to target concentration. In some embodiments, the non-responsive frequency is a frequency at which the signal is minimally-responsive to target concentration, i.e. the least responsive frequency across a range of frequencies. In some embodiments, the non-responsive frequency is a frequency at which sensor output is negatively-responsive to target concentration.

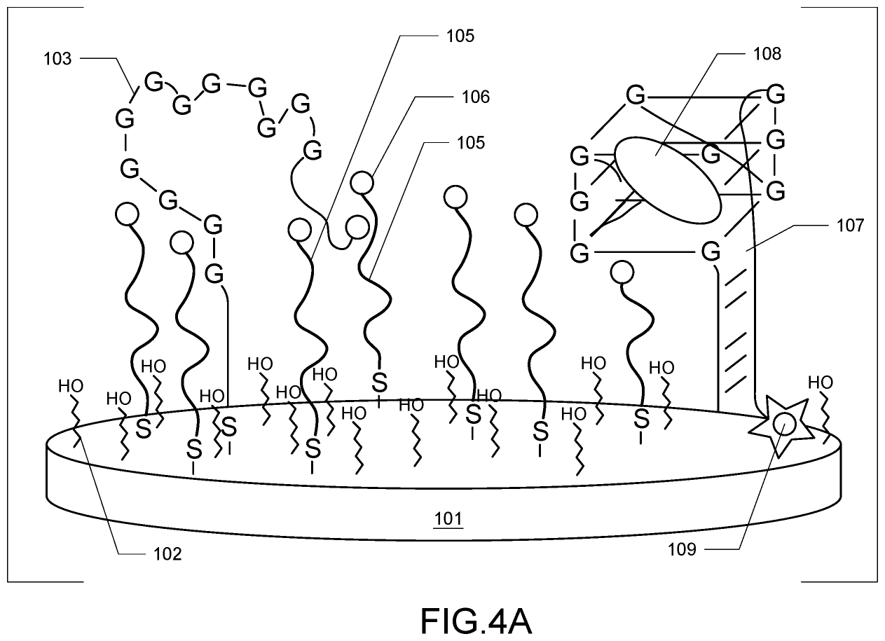

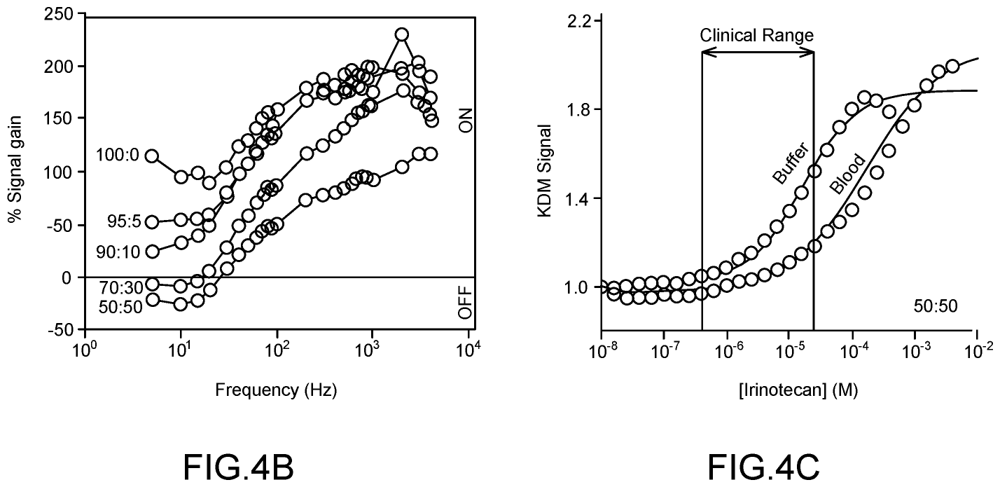

The mathematical relationship between drift corrected output and target concentration can be determined by one of skill in the art by routine experimentation for a particular sensor, or a particular sensor class, under a selected set of operating conditions, wherein sensor output is measured across a range of target concentrations, at the responsive and non-responsive frequencies to determine the mathematical relationship between drift corrected sensor output and target concentration in the sample. For example, a standard curve, a conversion factor, or any other mathematical relationship that relates the drift-corrected normalized signal to target concentration may be generated. For example, a drift-corrected normalized signal versus concentration plot as in FIG. 4C may be employed to relate drift corrected signal to target concentrations. For example, in one embodiment, a calibration curve is generated by exposing a sensor to a first calibration sample, for example, zero target, and then exposing the sensor to a series of calibration standards of different target concentration, and monitoring sensing and reference current to generate the standard curve across the dynamic range of the sensor.

Calibration Free Measurement. In some contexts, calibration-free measurement is desirable, for example, in the case of sensors deployed in vivo or other contexts wherein sensor calibration is impractical or burdensome. The novel sensors of the invention enable calibration-free measurement by providing a reference signal that is independent of the target concentration and which can be used in a ratiometric method to normalize signal output.

In various implementations, the scope of the invention encompasses a method of measuring the concentration of a target species in a sample by use of an electrochemical sensor, comprising the steps of:
obtaining measurements by means of a sensing element, wherein the sensing element comprises an electrode; wherein the electrode is functionalized with a plurality of a recognition element, wherein the recognition element is capable of selectively binding a target species and wherein the recognition element is functionalized with one or more redox reporters; and wherein the electrode is functionalized with a plurality of a charge transfer-modulating moiety,
exposing the sensing element to a calibration sample of known target concentration and (1) obtaining a baseline reference signal by applying a series of excitation pulses to the sensing element at a selected non-responsive frequency, wherein the non-responsive frequency is a frequency at which the signal is not measurably affected by the presence of target, is minimally responsive to the presence of target, or is negatively responsive to the presence of target; and (2) obtaining a baseline measurement signal by applying a series of excitation pulses to the sensing element at a selected responsive frequency, wherein the responsive frequency is a frequency at which the signal is dependent upon the concentration of target in the sample;
exposing the sensing element to a sample of unknown target concentration and (1) obtaining a reference signal by applying a series of excitation pulses to the sensing element at the non-responsive frequency; and (2) obtaining a measurement signal by applying a series of excitation pulses to the sensing element at the responsive frequency;
calculating a normalized reference signal, which is the ratio of the reference signal obtained in the sample of unknown target concentration to the baseline reference signal measured in the sample of known target concentration;
calculating the normalized measurement signal, which is the ratio of the measurement signal obtained in the sample of unknown target concentration to the baseline measurement signal obtained in the sample of known target concentration;
subtracting the normalized reference signal, which is indicative of drift, from the normalized measurement signal to obtain a normalized drift-corrected signal; and
applying a mathematical relationship between normalized drift-corrected signal and target concentration to the measured value of normalized drift-corrected signal to determine the target concentration in the sample.

The baseline calibration sample may be a sample of known target concentration. In one implementation, the baseline sample comprises a blank, i.e., a sample with zero target present, or in alternative implementation it can be a reference sample containing target at a known concentration.

It will be understood that the order of the measurement steps is not critical to the method, wherein reference and sensing signals can be obtained in any order and baseline and measurement signals may be collected in any order. In some embodiments, the measurement of baseline reference and sensing signals is performed prior to deployment of the sensor in a sample. For example, in one embodiment, the measurement of baseline sensing and reference signals is performed in a calibration solution and the sensor is subsequently deployed in vivo for collection of sensing and reference measurement signals.

In one embodiment, the normalized drift-corrected signal is determined as:

$${S}_{c o r} = \frac{{i}_{S}}{{i}_{S 0}} - \frac{{i}_{R}}{{i}_{R 0}} \qquad [ Equation 1 ]$$

wherein S_{cor} is the normalized, drift-corrected signal, is i_{S} the measurement signal measured in the sample of unknown concentration; i_{R} is the reference signal measured in the sample of unknown concentration; i_{S0} is the baseline sensing signal measured in the calibration sample of known concentration; and i_{R0} is the baseline reference signal measured in the calibration sample of known concentration.

The normalized drift-corrected change in signal can be converted to a target concentration value by comparing it against a standard curve, applying a conversion factor, or performing any other step that relates the measured normalized drift-corrected signal to target concentration.

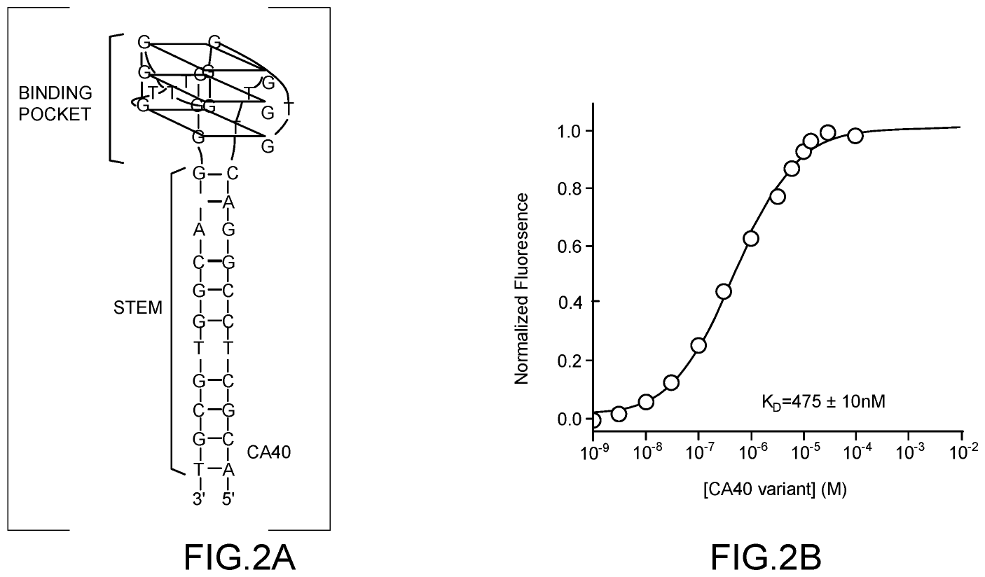

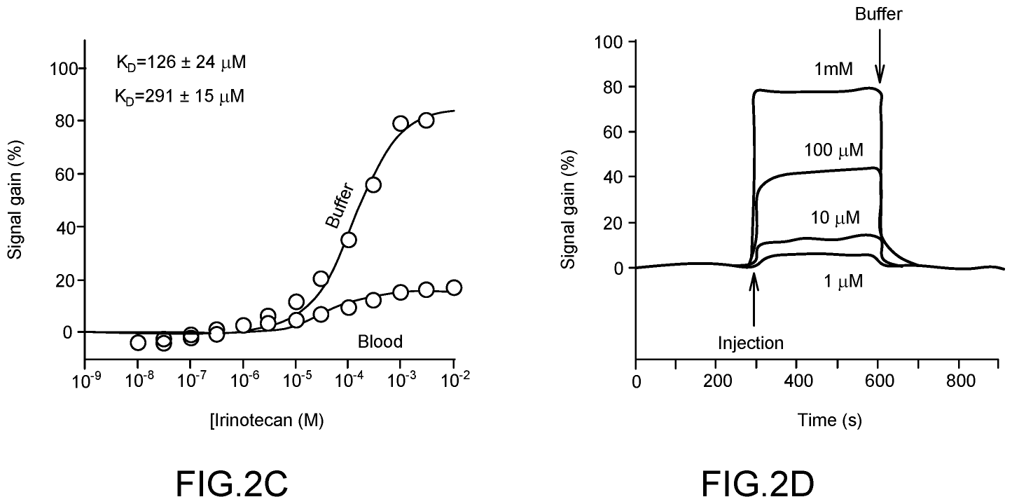

Improved Camptothecin Aptamers. In one embodiment, the scope of the invention comprises novel camptothecin binding aptamers. The camptothecin aptamer described in Fujita, et al., 2013. Efficacy of Base-Modification on Target Binding of Small Molecule DNA Aptamers, Pharmaceuticals 6: 1082-1093 is known in the art. A 40-base truncation of this aptamer, referred to herein as C40 comprises SEQ ID NO: 1, which folds into a target-recognizing G-quadruplex flanked by a 12-base-pair stem, as depicted in FIG. 2A The scope of the invention encompasses variants of the C40 aptamer.

In one embodiment, the camptothecin aptamer is CA40_1MM, comprising SEQ ID NO: 2 (5′-ACGCT CCGGACTTGG GTGGG TGGGT TGGGG TACGG TGTGT-3′), wherein nucleotides 1-13 and 29-40 make up the stem portion and nucleotides 14-28 make up G-quadruplex binding pocket.

In one embodiment, the camptothecin aptamer is CA40_2MM, comprising SEQ ID NO: 3 (5′-ACGCT CTGGA CTTGG GTGGG TGGGT TGGGG TACGG TGTGT-3′) wherein nucleotides 1-13 and 29-40 make up the stem portion and nucleotides 14-28 make up G-quadruplex binding pocket.

In one embodiment, the camptothecin aptamer is CA36, comprising SEQ ID NO: 4 (5′-GCTCC GGACT TGGGT GGGTG GGTTG GGGTA CGGTG C-3′) nucleotides 1-11 and 28-36 make up the stem portion and nucleotides 12-27 make up the G-quadruplex binding pocket.

In one embodiment, the camptothecin aptamer is CA32, comprising SEQ ID NO: 5 (5′-TCCGG ACTTG GGTGG GTGGG TTGGG GTACG GT-3′), wherein nucleotides 1-9 and 26-32 make up the stem portion and nucleotides 1-25 make up the G-quadruplex binding pocket.

In one embodiment, the camptothecin aptamer is CA28, comprising SEQ ID NO: 6: (5′-CGGAC TTGGG TGGGT GGGTT GGGGT ACG-3′), wherein nucleotides 1-7 and 24-28 make up the stem portion and nucleotides 8-23 make up the G-quadruplex binding pocket.

In one embodiment, the camptothecin aptamer is CA16, comprising SEQ ID NO: 7 (5′-GGGTGGGTGGGTTGGG-3′), comprising only a G-quadruplex binding pocket.

In one embodiment, the scope of the invention comprises a sensor for the measurement of camptothecin and derivatives thereof, comprising a recognition element comprising a camptothecin aptamer selected from the group consisting of: C40 (SEQ ID NO: 1); CA40_1MM, (SEQ ID NO: 2); CA40_2MM (SEQ ID NO: 3); CA36 (SEQ ID NO: 4); CA32 (SEQ ID NO: 5); CA28 SEQ ID NO: 6); and CA16 (SEQ ID NO: 7).

In one embodiment, the scope of the invention encompasses a method of measuring the concentration of a camptothecin compound in a sample, comprising the use of an electrochemical sensor comprising a recognition element comprising a camptothecin aptamer selected from the group consisting of: C40 (SEQ ID NO: 1); CA40_1MM, (SEQ ID NO: 2); CA40_2MM (SEQ ID NO: 3); CA36 (SEQ ID NO: 4); CA32 (SEQ ID NO: 5); CA28 SEQ ID NO: 6); and CA16 (SEQ ID NO: 7). The camptothecin compound may be camptothecin or a derivative thereof, for example, irinotecan, topotecan, belotecan, or deruxtecan.

### EXAMPLES

### Example 1. Seconds-Resolved Pharmacokinetic Measurements of the Chemotherapeutic Irinotecan In Situ in the Living Body

Introduction. The goal of personalized medicine is to precisely tailor treatment to the individual. To this end, an ability to measure drugs in the living body with seconds resolution would allow clinicians to determine drug dosing based on high-precision, patient-specific pharmacokinetic measurements rather than on indirect predictors of drug metabolism such as age, body mass, or pharmacogenetics. Ultimately, the ability to measure drugs in the body in real-time would enable closed-loop feedback-controlled delivery, vastly improving dosing precision by actively responding to minute-to-minute fluctuations in a patient's metabolism. The development of such technology, however, faces significant hurdles. First, an in vivo sensor must be small enough to be placed in the body without causing undue damage. Second, it cannot require the addition of exogenous reagents or the use of batch processing, such as washing or separations. Third, it must make measurements at a frequency that is rapid relative to the drug's pharmacokinetics. Finally, it must be selective and stable enough to work for prolonged periods in the complex, fluctuating environments found in vivo. To this end described herein is an improved electrochemical aptamer-based (E-AB) sensor.

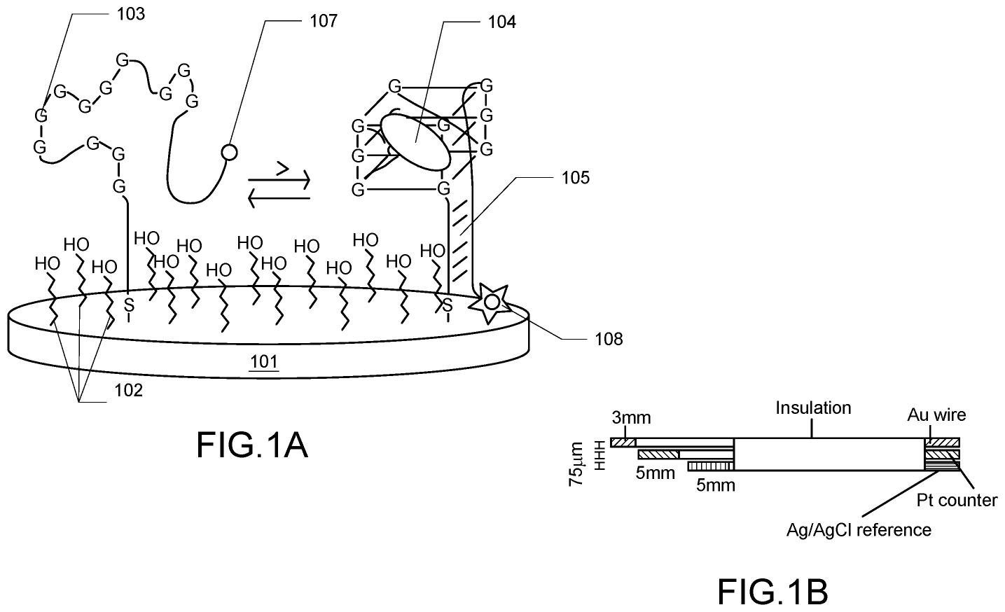

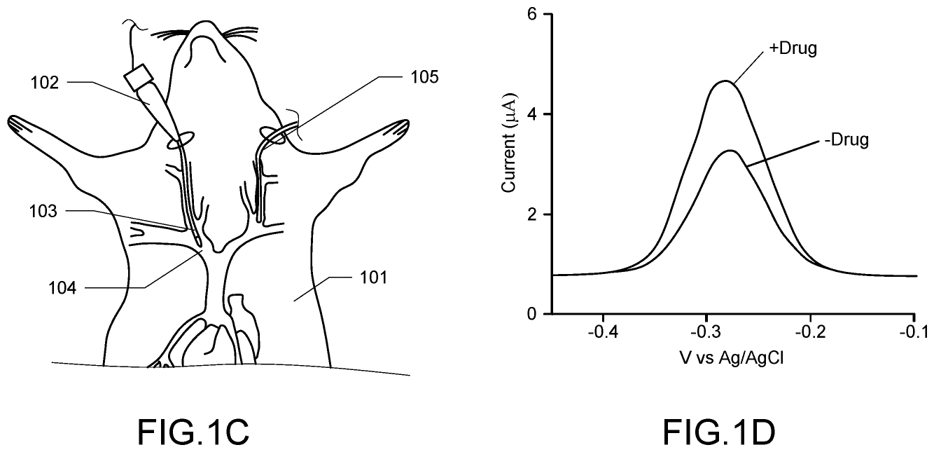

E-AB sensors employ an electrode-bound, redox-reporter-modified aptamer as their recognition element (FIG. 1A). Binding of the target molecule to this aptamer induces a conformational change that produces an easily measured electrochemical output (for example, square wave voltammetry) without needing reagent additions or wash steps. Because E-AB signaling is generated by a binding-induced conformational change and not, as is the case for most other reagentless biosensor architectures, by the adsorption of target to the sensor surface, E-AB sensors are largely insensitive to non-specific adsorption and support multi-hour measurements in biological fluids not only in vitro but also in vivo. Finally, because their signaling arises due to target binding alone, and not, as is the case, for example, of the continuous glucose monitor, from the chemical reactivity of the target, E-AB sensors are a platform technology generalizable to a wide variety of analytes, including two of which, the aminoglycosides and doxorubicin, have been measured in vivo. Building on this foundation, described herein is the fabrication and characterization of an E-AB sensor adapted to measurements in situ in the body, one directed against the camptothecin family of anti-cancer drugs, an important class of chemotherapeutic agents used in the treatment of a range of human cancers.

Results and discussion. As the recognition element in the sensor, a DNA aptamer that binds to the camptothecins was employed. Specifically, a 40-base version of the aptamer, termed CA40, which folds into a target-recognizing G-quadruplex flanked by a 12-base-pair stem (FIG. 2A), binds the camptothecin, irinotecan, with a dissociation constant of about 475 nM when the unmodified aptamer is free in solution (FIG. 2B). To adapt this into an E-AB sensor, its 3′ end was modified with a methylene blue redox reporter and deposited it onto a gold electrode via a six-carbon thiol at its 5′ end (FIG. 1A). Electrochemically interrogating the resulting sensor in buffer, the expected Langmuir isotherm binding was observed with an estimated K_{D} of 126+/−24 mM, signal gain (the relative change in signal upon the addition of saturating target) of 84 4% (FIG. 2C,) and association and dissociation kinetics too rapid to measure (time constants <5 s at clinically relevant concentrations; FIG. 2D). The poorer affinity the aptamer exhibits in the context of the sensor likely arises due to interactions with the electrode surface, as this is known to destabilize the folding (thus hindering binding) surface-attached oligonucleotide. Despite this, when challenged in buffer the sensor supports the detection of irinotecan over the clinically relevant 1 to 15 mM range. When challenged in whole blood, however, the (apparent) affinity of the surface-bound aptamer is poorer still (K_{D}=291+/−15 mM), presumably because the concentration of the free drug is reduced due to protein binding. Worse, under these conditions the gain falls to about 15%, pushing its useful dynamic range out of the clinically relevant concentration window (FIG. 2C).

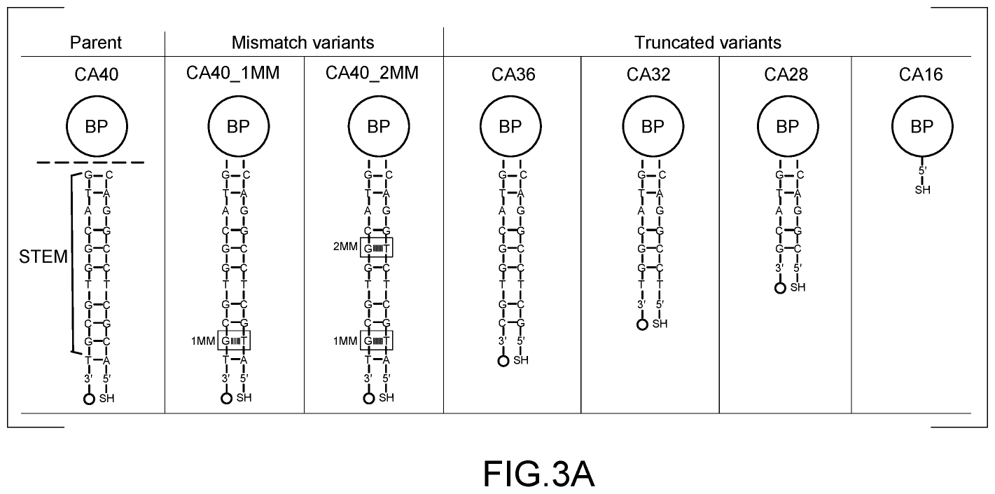

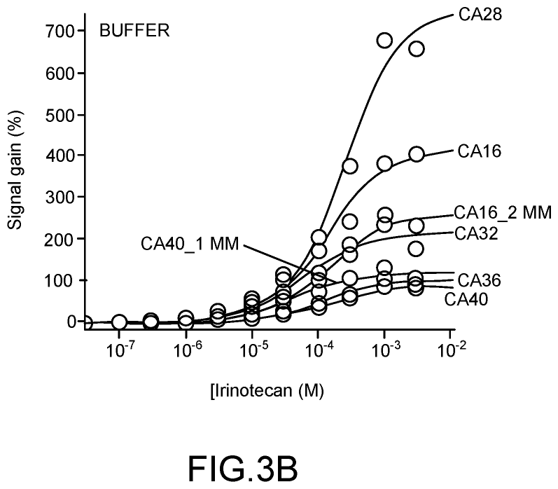

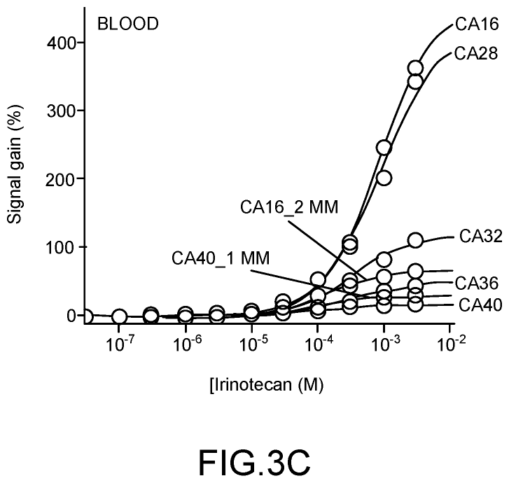

To improve sensor performance in whole blood, the camptothecin-binding aptamer was reengineered to better populate its “unfolded” state in the absence of target, thus increasing the sensor's gain. To do so the aptamer's double-stranded stem was destabilized (FIG. 3A) via either truncation (CA36, CA32, CA28 CA16) or the introduction of one (CA40_2MM) or two (CA40_2MM) G-T mismatches. As estimated using the nucleic acid folding predictor NUPACK software. These strategies should decrease the stability of folded aptamer from the 31.8 kJ mol⁻¹ of the C40 parent to as low as 0.3 kJ mol¹ for CA28. Characterizing sensors fabricated using these variants (FIG. 3A) dissociation constants were obtained ranging from 38 11 mM to 254 48 mM and signal gain of up to 755% (FIG. 3B) when challenged in working buffer. Testing in whole blood (FIG. 3C) once again reduces both gain and apparent affinities. Even under these more challenging conditions, however, sensors employing the CA32, CA28, and CA16 variants supported high-gain E-AB sensing.

Having achieved good in vitro performance with a sensor employing the CA32 variant, next the sensor was adapted to in situ use in the veins of live rats. Under such conditions, E-AB sensors often exhibit significant baseline drift. Previously this has been corrected using “Kinetic Differential Measurements” an approach that exploits the generally strong square-wave frequency dependence of E-AB signal gain. Specifically, the signal gain of the E-AB sensors previously described is so great that they exhibit a “signal-on” (target binding increases the signaling current) response at some square-wave frequencies no observable gain or even “signal-off” behavior at others. Conveniently, the signals obtained under these different regimes drift in concert such that taking their difference via KDM removes the drift seen in vivo. And since the two signaling currents respond in opposition in the presence of their target, taking their difference also improves signal gain.

In contrast, the gain of the camptothecin-detecting E-AB sensors (CA32, CA28, and CA16) is only a weak function of square-wave frequency, necessitating the development of a new approach to performing KDM. To enhance the frequency-dependence of the sensor's gain in support of KDM drift correction we added a second reporter-modified DNA strand to the sensor that: (1) transfers electrons more rapidly than the aptamer does and (2) does not respond to the presence of the target. The rational for doing so was that, at frequencies at which this “non-responsive” DNA dominates the signal (i.e., at higher frequencies) the gain of the resultant sensor will be low, and at frequencies at which, instead, the aptamer dominates the signal gain will be higher. To achieve this the CA32 aptamer variant was co-deposited with and a secondary moiety comprising an unstructured 10-base strand (SEQ ID NO: 8) comprised of a random sequence of adenines and thymines that is known to transfer electrons at a rate of 80 per second. Per expectations, sensors employing a 1:1 mixture of this sequence and the aptamer achieve sufficiently frequency-dependence gain to enable KDM (FIG. 4B). Surprisingly, the resultant frequency dependence is so strong that the sensor's gain becomes slightly negative at low frequencies, an observation inconsistent with the expectations described above (if the currents are additive, the gain cannot go below zero). This is presumed this occurs due to interactions between the two sequences on the surface that alter their electron transfer kinetics. Irrespective of its origins, however, the effect supports accurate KDM drift correction. Specifically, using the signals obtained at 10 Hz (signal-off) and 120 Hz (signal-on) to perform KDM, irinotecan can be monitored in both buffer and whole blood (FIG. 4C) over the entire 0.5 to 15 mM (0.06 to 10 mg per mL) human therapeutic range.

KDM-corrected indwelling E-AB sensors readily support the real-time, high frequency irinotecan measurements in situ in the bodies of live rats. Sensors using 75 mm-diameter gold, platinum and silver wires as the working, counter and reference electrodes, respectively were fabricated (FIG. 1). Sensors were placed in the jugular vein of anesthetized Sprague-Dawley rats via a previously emplaced 22-gauge catheter (FIG. 1C). Testing this with a single intravenous injection (20 mg per kg) of irinotecan the signal observed at both 10 and 120 Hz responded to the drug, but was also accompanied by the expected signal drift. The gain observed at 10 Hz becomes, under these conditions, slightly positive. KDM was able to be used to correct the sensor's drift and recover stable baselines, thus enabling continuous, real-time measurements of the drug at therapeutically relevant concentrations.

To further characterize the performance of the camptothecin sensor, it was used it to monitor sequential intravenous injections of irinotecan (at 10 and 20 mg kg). The resultant maximum concentrations (C_{MAX} ¼ 39.8 3.2 mM and 20.9+/−2.0 mM, respectively; here and below the confidence intervals reflect standard errors calculated from the fits) and distribution rates (a V4 0.58+/−0.07 min and 0.48 min) are comparable to those seen in previous studies that employed ex-vivo drug-level measurements. In contrast, the elimination rates (b V4 9.2+/−1.4 min and 8.4+/−2.2 min) observed are more rapid than those reported previously and, thus, the resulting “areas under the curve” for the drug are reduced. It is believed that this discrepancy occurred because the prior work used chromatographic and mass spectrometric methods to measure total drug levels (which requires removal of blood samples from the animal's body and the extraction of the total drug into buffer). E-AB sensors, in contrast, measure the free drug, which is the fraction of the drug that is pharmacologically active. And, in general, the elimination and clearance of free drug are more rapid than those of total drug as drugs that interact strongly with plasma proteins tend to clear more slowly than those that do not.

E-AB-derived measurements of irinotecan pharmacokinetics represent a significant advance over prior pharmacokinetic studies of the camptothecins. For example, the 20 s temporal resolution of our measurements (defined by the time required to take the two square wave scans necessary to perform KDM) is at least an order of magnitude better than that of the most highly time resolved prior study. Moreover, all prior studies reported plasma level measurements averaged over multiple animals, thus eliminating their ability to explore subject-to-subject pharmacokinetic variability. The present E-AB-derived measurement parameters, in contrast, provide 300 time points per hour in each animal, and thus determine the pharmacokinetics of individuals with exceptional precision. Because the excretion phase of irinotecan exhibits significant inter-patient variability (due to drug-drug interactions, variations in health status, and pharmacogenetics), this latter point is likely of clinical significance. To illustrate the ability to measure such variability we performed sequential 10 and 20 mg per kg irinotecan injections in rats (FIG. 7, representative data from single rat). The resulting measurements reveal only small (10 to 20%) variation in either CMAX or the rate of the distribution phase. In contrast, however, the rate of drug excretion and its clearance values varied many fold from individual to individual rat. As all of the animals we employed in these experiments were healthy male rats these pharmacokinetic differences arose solely due to metabolic variability between the animals.

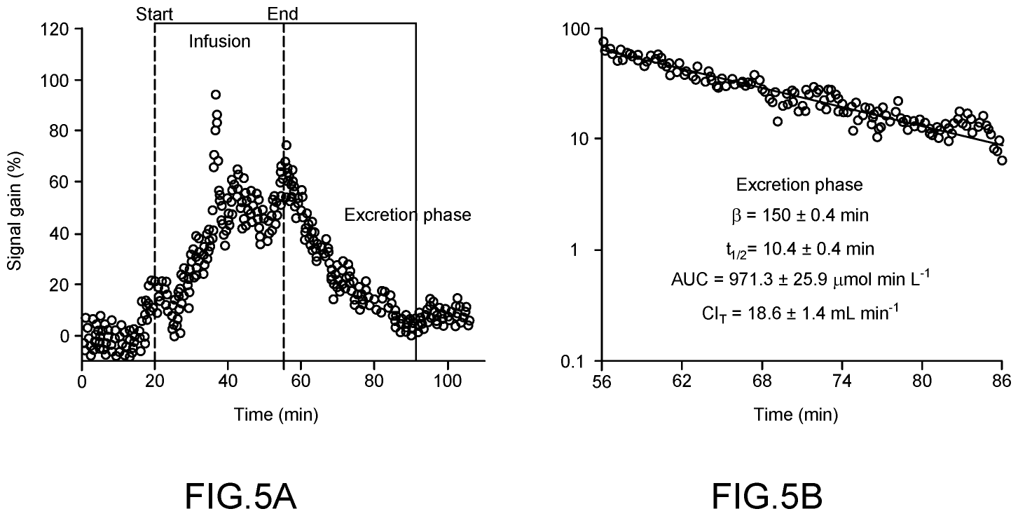

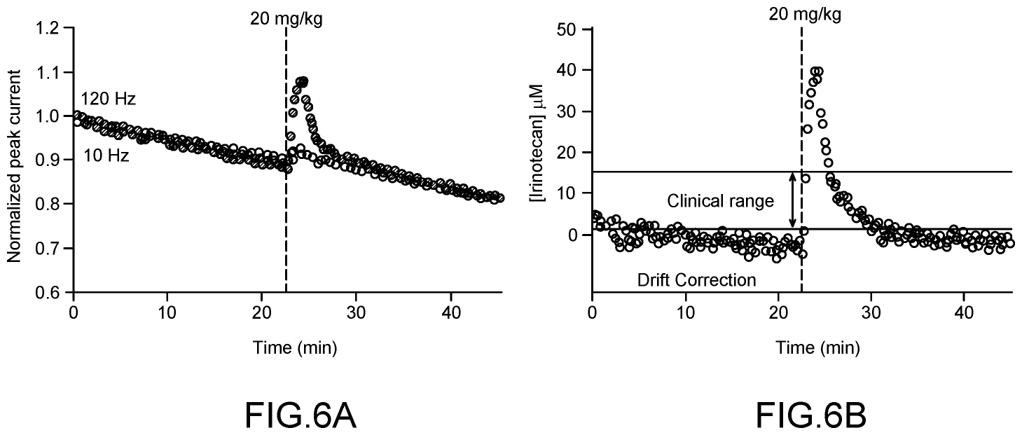

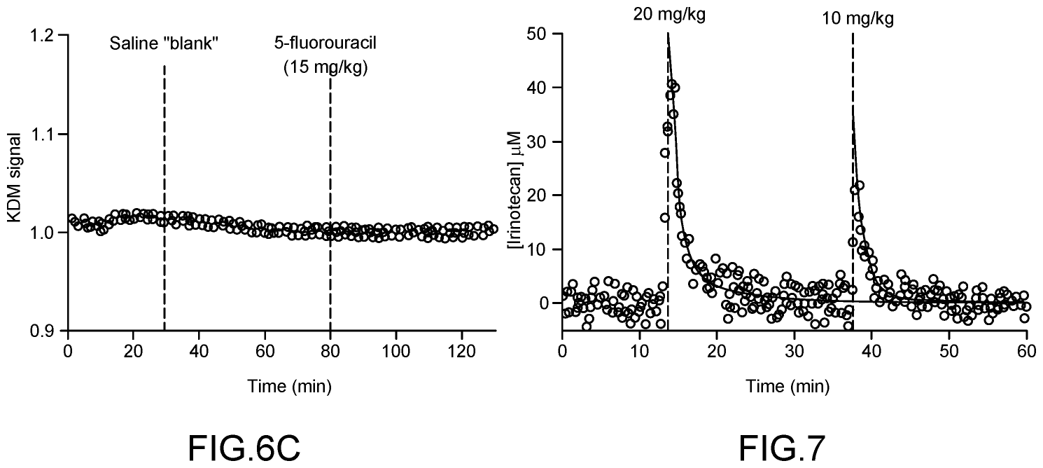

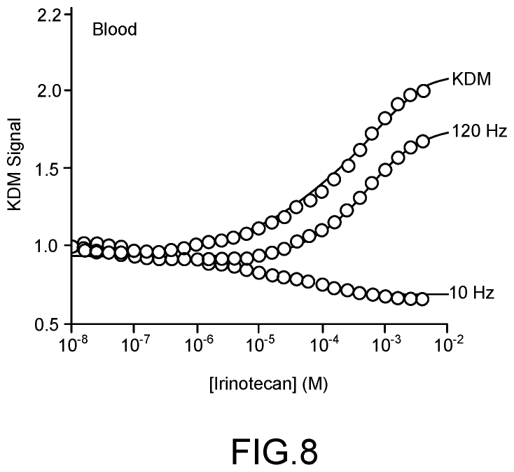

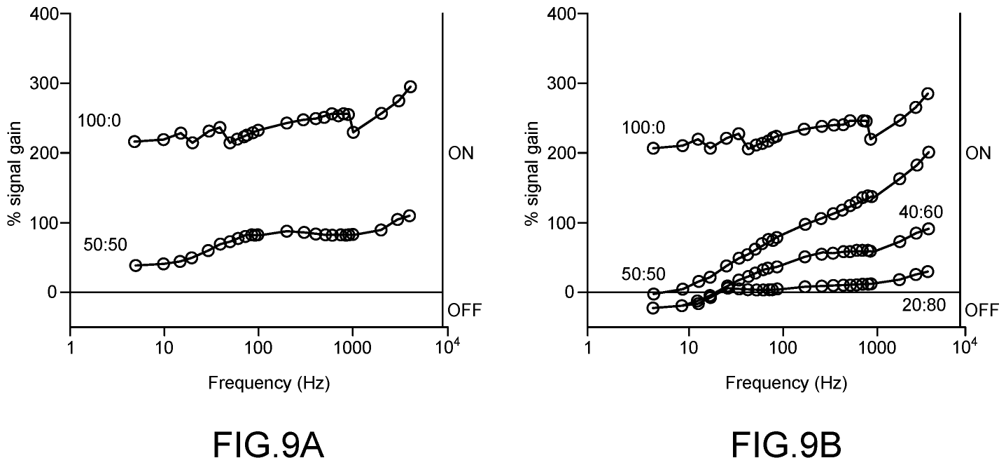

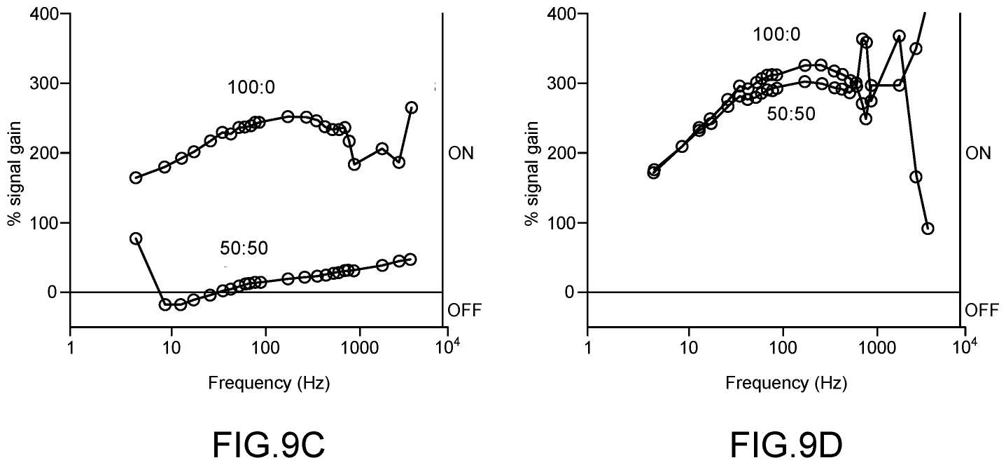

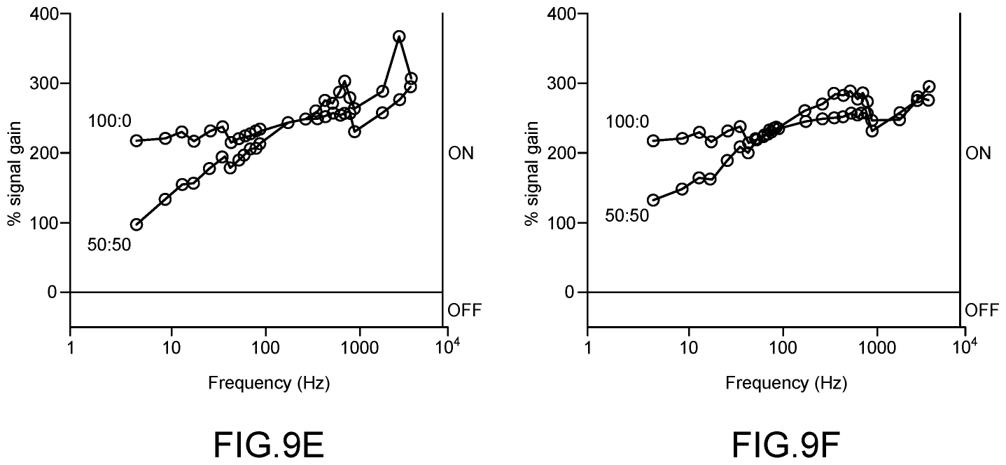

The elimination rate and clearance of irinotecan are the pharmacokinetic parameters used to determine its optimized, personalized dosing during chemotherapy. To measure these parameters with greater precision we administered a large dose (60 mg per kg) of the drug over a longer period. The higher plasma concentrations this produces lead, in turn, to a longer measurement period (after delivery ceases) before the sensor's limit of detection is reached (FIGS. 5A and 5B). Thus the 150 measurements achieved in a single pharmacokinetic profile produced estimates of the drug's elimination half-life (10.4+/−0.4 min) and clearance (18.6+/−1.4 mL per min) that are far more precise than those produced in prior, ex vivo studies, which typically achieve less than a dozen measurements per profile, much less the two measurements used in typical “peaks-and-troughs” clinical measurements.

Here is describe an indwelling E-AB sensor supporting the seconds-resolved measurement of the anticancer drug irinotecan in situ in the living body over the course of hours. Design of the sensor required the reengineering of a parent aptamer to support high-gain E-AB signaling and the development of a novel method to ensure sufficient frequency-dependent signal gain to support KDM-based drift correction. Using the resulting sensors plasma irinotecan levels were measured with micromolar concentration resolution and seconds temporal resolution, with the latter representing an orders of magnitude improvement over that of prior studies. The resulting measurements define the pharmacokinetics of irinotecan of individual animals, providing an unprecedented high precision view of the drug's inter-subject pharmacokinetic variability.

E-AB sensors are a platform technology that supports the high frequency, real-time measurement of specific molecules (irrespective of their chemical reactivity) in situ in the living body. When coupled with the platform's convenience and precision this versatility provides significant opportunities to improve drug dosing. As noted above, for example, irinotecan suffers from significant inter-patient metabolic variability, leading to toxicity and increasing side effects. But because current methods for measuring plasma drug levels are slow and cumbersome, the FDA has invoked pharmacogenetic estimates of metabolism as the primary means of reducing the risk associated with this variation. In this light, the ease with which E-AB sensors provide high precision, patient-specific measurements of drug elimination (as opposed to indirect estimates), suggests the platform could provide a valuable adjunct to chemotherapeutic treatment.

In addition to improving the precision and accuracy of personalized dose determination, E-AB-derived measurements may also support a new paradigm for personalized drug delivery. Specifically, the real-time concentration information provided by E-AB sensors may be used to inform closed-loop feedback controlled drug delivery. In this the rate of drug administered is optimized multiple times a minute, enabling the maintenance of plasma drug concentrations at a pre-defined value with precision of better than 20% despite 3-fold hour-to-hour changes in drug pharmacokinetics. This approach to drug delivery provides an unprecedented means of overcoming pharmacokinetic variability, improving the overall efficacy and safety of treatment. Given this, by the improvements of the present invention, drug-detecting E-AB sensors provide a powerful new tool in the clinician's arsenal.

Materials and Methods

Sensor Fabrication. Catheters (22 G) and 1 mL syringes were used. Polytetrafluoroethylene-insulated gold, platinum, and silver wires (75 m diameter) were used. To employ the silver wires as reference electrodes they were immersed in concentrated sodium hypochlorite (commercial bleach) overnight to form a silver chloride film. Heat-shrink polytetrafluoroethylene insulation was used to electrically insulate the wires. Custom-made, open ended, mesh-covered three channel connector cables to fabricate in vivo probes were used.

Oligonucleotides. For sensor fabrication the aptamer variants were purchased modified with a thiol-C6-SS group at its 3′ end, and a methylene blue attached by a six-carbon linker to an amine at its 5′ end. The oligonucleotides were dissolved in buffer (100 mM Tris buffer, 10 mM MgCl₂, pH 7.8) at a concentration of 100 μM and then aliquoted and stored at −20° C.

Electrode Polishing and Cleaning. The E-AB sensors employed in-vitro were fabricated using established approaches, for example, as described in B. Sanavio and S. Krol, Front. Bioeng. Biotechnol., 2015, 3, 20. Briefly, E-AB sensors were fabricated on rod gold disk electrodes. The disk electrodes were prepared by polishing on a microcloth pad soaked with a 1 m diamond suspension slurry (and then with a 0.05 m alumina powder aqueous suspension. Each polishing step is followed by sonication of the electrodes in a solution 1:1 water/ethanol for 5 min. The electrodes were then electrochemically cleaned following this procedure: a) The electrodes are placed in a 0.5 M NaOH solution and through cyclic voltammetry 1000-2000 scans are performed using a potential between −0.4 and −1.35 V versus Ag/AgCl at a scan rate of 2 V per s; b) The electrodes were moved to a 0.5 M H₂SO₄ solution and using chronoamperometry an oxidizing potential of 2 V was applied for 5 s. After a reducing potential of −0.35 V was then applied for 10 s c) Using cyclic voltammetry the electrodes were cycled rapidly (4 V per s) in the same solution between −0.35 and 1.5 V for 10 scans followed by 2 cycles recorded at 0.1 V per s using the same potential window.

Electrode functionalization. For the E-AB sensors functionalized with a classic monolayer composed by a single DNA probe the DNA probe was first reduced (100 μM) by treating it for 1 hour in a solution of 10 mM tris(2-carboxyethyl)-phosphine hydrochloride (TCEP) at room temperature in the dark. This was then dissolved in “assembling buffer” (10 mM Na₂HPO₄ with 1 M NaCl and 1 mM MgCl₂ at pH 7.3) at a final concentration of 500 nM. The electrochemically cleaned gold electrodes were then immersed in 200 μL of this solution for 1 hour in the dark. Following this the electrode surface was rinsed with distilled water and incubated overnight at 4° C. in assembling buffer containing 5 mM 6-mercaptohexanol, followed by a further rinse with distilled water before use. For the E-AB sensors functionalized with a mixed monolayer formed by CA32 aptamer and the linear probe, the two DNA probes were first reduced (100 μM) by treating them for 1 hour in a solution of 10 mM tris(2-carboxyethyl)-phosphine hydrochloride (TCEP) at room temperature in the dark. These were then dissolved in buffer (10 mM Na₂HPO₄ with 1 M NaCl and 1 mM MgCl₂ at pH 7.3) using different ratio of the two DNA probes. The total oligonucleotide concentration was kept constant at 500 nM to maintain a constant DNA probes packing density. The electrochemically cleaned gold electrodes were then immersed in 200 μL of this solution composed of two DNA probes for 1 hour in the dark. Following this step, the electrode surface was rinsed with distilled water and incubated overnight at 4° C. in assembling buffer containing 5 mM 6-mercaptohexanol, followed by a further rinse with distilled water before use.

In-vivo Sensors: Electrode fabrication. The E-AB sensors employed in-vivo were fabricated as described in previous reports (Tucker, Pharm. Res., 2017, 34, 1539-1543, N. Arroyo-Curras, G. Ortega, D. A. Copp, K. L. Ploense, Z. A. Plaxco, T. E. Kippin, J. P. Hespanha and K. W. Plaxco, ACS Pharmacol. Transl. Sci., 2018, 1, 110-118). Segments of pure gold (7.75 cm in length), platinum (7.25 cm), and silver (6.75 cm) wire, were cut to make sensors. The insulation at both ends of these wires, about 1 cm, was removed using a surgical blade to allow electrical contact. These were then soldered each to one of the three ends of a connector cable using 60% tin/40% lead rosin-core solder (0.8 mm diameter) and then attached together by applying heat to shrinkable tubing around the body of the wires, except for a small window of about 5 mm at the edge of each wire. The wires were attached in a layered fashion, with the gold wire being insulated alone first, then both gold and platinum wires together, and finally all three wires together. The purpose of this three-layer-thick insulation was to give mechanical strength to the body of the malleable probe. To prevent electrical shorts between wires, different lengths were used for each wire as described above. The sensor window (i.e., the region devoid of insulation) in the gold wire was cut to approximately 3 mm in length. To increase the surface area of the gold working electrodes (to obtain larger peak currents) the sensor surface was roughened electrochemically via immersion in 0.5 M sulfuric acid followed by stepping the potential between Eᵢₙᵢₜᵢₐₗ=0.0 V to E_{high}=2.0 V vs Ag/AgCl, back and forth, for 16 000 pulses. Each potential step was of 20 ms duration with no wait time between pulses.

Electrode functionalization. The E-AB sensors employed in-vivo were functionalized with a mixed monolayer formed by CA32 aptamer and the linear probe SEQ ID NO: 8. The two DNA probes (100 μM) were first reduced by treating them for 1 hour in a solution of 10 mM tris(2-carboxyethyl)-phosphine hydrochloride (TCEP) at room temperature in the dark. These were then dissolved in “assembling buffer” (10 mM Na₂HPO₄ with 1 M NaCl and 1 mM MgCl₂ at pH 7.3) employing a 1:1 mixture of the two DNA probes (i.e. 250 nM for each DNA probe) for a total oligonucleotides concentration of 500 nM. The electrochemically roughened gold electrode was rinsed in deionized water before being immersed in 200 μL of this solution composed by two DNA probes for 1 hour in the dark. Following this the electrode surface was rinsed with distilled water and incubated overnight at 25° C. in assembling buffer containing 5 mM 6-mercaptohexanol, followed by a further rinse with distilled water before use.

In-vitro measurements. Electrochemical measurements were performed at room temperature using a CHI660D potentiostat with a CHI684 Multiplexer (CH Instruments, Austin, Tex.) and a standard three-electrode cell containing a platinum counter electrode and a Ag/AgCl (3 M KCl) reference electrode. Square Wave Voltammetry (SWV) was performed using a potential window of −0.1 to −0.4 V, a potential step of 1 mV and an amplitude of 50 mV.

Titration curves. Experimental titration curves were performed in 10 mL of working buffer (137 mM NaCl, KCl 2.7 mM, Na₂HPO₄ 10 mM, KH₂PO₄ 1.8 mM at pH 7.3) using three E-AB sensors modified with the oligonucleotide probe and using a SWV frequency of 10 and 120 Hz. Initially, in absence of irinotecan, we performed a preliminary treatment by interrogating the electrodes with 30-60 scans until a stable current peaks were obtained. Once the sensor's signal was stable increasing concentrations of the irinotecan was added and the sensors were interrogated after 10 min. The electrochemical signal (peak current) of each sensors was plotted in function of irinotecan concentrations and then it was fitted using a single-site binding mechanism equation:

$${C}_{R a w}^{[ Irinoteca n ]} = {C}_{R a w}^{0} + ( \frac{[ Irin otecan ] ( {C}_{MAX}^{[ Irinotecan ]} - {C}_{R a w}^{0} )}{[ Irin otecan ] + {K}_{D}} ) \qquad Equation 2$$

Where [Irinotecan] is the irinotecan concentration, C^{[Irinotecan raw]} is signal current in the presence different concentrations of irinotecan, C^{0 raw} is the background current seen in the absence of the irinotecan, C^{[Irinotecan]} is the current signal seen at saturating concentrations of irinotecan, and KD is the dissociation constant of the surface-bound aptamer. Using the values estimated from the previous fitting procedure the raw signal current was converted in signal change % (C_{%}) using the following equation:

$${C}_{\%} = ( \frac{{C}_{R a w}^{[ Irinotecan ]} - {C}_{R a w}^{0}}{{C}_{R a w}^{0}} ) . \qquad Equation 3$$

The binding curves performed in whole blood were conducted following the previous described experimental approach but using a closed-loop system with a continuous flow of whole blood (total volume of 20 mL at 1 mL/s) through a circulator pump.

Sensor equilibration time. The sensor's equilibration time was determined using the above experimental approach and interrogating the sensor every 5 s in working buffer and using a SWV frequency of 500 Hz. After a stable current baseline (5 min) was reached, different concentrations of irinotecan were added to the solution (from 1 μM to 1 mM) and the voltammetric signal as monitored for over 5 min. Then the sensor was moved in simple working buffer without irinotecan and the voltammetric signal was collected for over 5 min. The observed signal change was fitted to a single exponential decay to obtain the equilibration time constant of the sensor.

Square wave voltammetry frequency vs signal change (%) plot. The dependence of E-AB sensors's signal was calculated as change (%) on the square wave voltammetry frequencies interrogating the sensors using various frequencies (from 5 Hz to 4000 Hz) in absence and in presence of a saturating amount of irinotecan (1 mM and 100 μM, and an incubation time of 10 min). The collected peaks current, in absence and in presence of a saturating amount of irinotecan (1 mM or 100 μM), were converted in signal change % using the equation above. Then the estimated signal change (%) was plotted in function of the relative square wave frequency.

In-vivo measurements. All in vivo measurements were performed using a three-electrode setup in which the counter electrodes were made of platinum wire and the reference electrodes were a silver wire coated with a silver chloride film as described above. The measurements carried out in vivo were recorded using a handheld potentiostat from CH Instruments (Model 1242 B). Square Wave Voltammetry (SWV) was performed using a potential window of −0.1 to −0.45 V, a potential step of 0.001 V and 0.05 V amplitude

For in vivo measurements a 30 min sensor baseline was established before the drug infusion. A 3 mL syringe filled with the target drug was connected to the sensor-free catheter (placed in the jugular opposite that in which the sensor is emplaced) and placed in a motorized syringe pump. After establishing a stable baseline, the drug was infused through this catheter at a rate of 0.6 mL/min using irinotecan at 15 mg/mL or 5-fluorouracil at 10 mg/mL. After drug infusion, recordings were taken for up to 1 hour before the next infusion. The real-time plotting and analysis of voltammetric data were carried out with the help of a script written in Python.

Animals. In vivo measurements were performed in male and female Sprague-Dawley rats (4-5 months old) weighing between 300 and 500 g. All animals were pair housed in a standard light cycle room (08:00 on, 20:00 off) and allowed ad libitum access to food and water. For in vivo measurements rats were induced under 5% isoflurane anesthesia in a Plexiglas anesthesia chamber. The rats were then maintained on 2-3% isoflurane gas for the duration of the experiment. A small incision was made above each vein, then each vein was isolated. A small hole was cut into each vein with spring-loaded microscissors. The silastic catheter inserted into the left jugular vein was constructed with a bent steel cannula with a screw-type connector and silastic tubing (11 cm, i.d. 0.64 mm, o.d. 1.19 mm). The EAB sensor was constructed as previously mentioned and inserted into the right jugular vein. Both the E-AB sensor and the infusion line were tied into place with sterile 6-0 silk suture, then 30 units of heparin.

Pharmacokinetic Analysis. Plasma irinotecan concentration-time curves were fitted to a bi-exponential equation describing a two-compartment model:

$$[ Irinotecan ] = A {e}^{- t / α} + B {e}^{- t / β} \qquad Equation 4$$

where A and B are amplitudes, and a and R are the half-lives for distribution and elimination, respectively. To improve the precision of the fitting a time range of 20 minutes was used for the pharmacokinetic curves obtained using an intravenous dose of 20 mg/kg of drug, and a time range of 10 minutes for the curves obtained using an intravenous dose of 10 mg/kg of irinotecan. Moreover, to improve the precision of the fitting equation α and β were fixed for the rat 1 and rat 3 for the curves obtained using an intravenous dose of 10 mg/kg of irinotecan. The peak concentration (CMAX) was directly obtained from the experimental values of each irinotecan concentration-time curves. The area under the curve (AUC) was calculated using the parameters A, B, α and β, because integrating the equation 5 from 0 to infinity, it holds:

$$AUC = \frac{A}{( \frac{1}{1 / α} )} + \frac{B}{( \frac{1}{1 / β} )} \qquad Equation 5$$

The clearance (Cl_{T}) was calculated by the equation:

$$C {l}_{T} = \frac{Dose}{AUC} \qquad Equation 6$$

The errors for C_{MAX}, AUC and Cl_{T} are propagated from the errors associated to the kinetic parameters A, B, α and β.

NUPACK Simulations. NUPACK software (http://www.nupack.org/) was used to predict the folding free energies of the stem portion for the CA40 aptamer variants The aptamer sequences were analyzed by the software using the following parameters: (a) temperature: 25° C.; (b) number of strand species: 1; (c) maximum complex size: 4; (d) oligo concentration=500 nM; in advanced options; (e) [Na+]=0.15 M, [Mg++]=0 M; (f) dangle treatment: some.

All patents, patent applications, and publications cited in this specification are herein incorporated by reference to the same extent as if each independent patent application, or publication was specifically and individually indicated to be incorporated by reference. The disclosed embodiments are presented for purposes of illustration and not limitation. While the invention has been described with reference to the described embodiments thereof, it will be appreciated by those of skill in the art that modifications can be made to the structure and elements of the invention without departing from the spirit and scope of the invention as a whole.

1. A sensing element
wherein the sensing element is configured for use in an electrochemical sensor for measuring the concentration of a target species in a sample, comprising
an electrode; wherein the electrode is functionalized with a plurality of a recognition element, wherein the recognition elements are capable of selectively binding a target species and wherein the recognition elements are functionalized with one or more redox reporters; and
wherein the electrode is functionalized with a plurality of a charge transfer-modulating moieties, wherein the charge-transfer modulating moieties do not substantially bind to the target species.

2. The sensing element of claim 1, wherein
the recognition element comprises a polynucleotide, a polypeptide, or a chemical species.

3. The sensing element of claim 2, wherein
the recognition element comprises a polynucleotide.

4. The sensing element of claim 3, wherein
the polynucleotide comprises an aptamer.

5. The sensing element of claim 1, wherein
the charge transfer-modulating moiety comprises an oligonucleotide.

6. The sensing element of claim 5, wherein
the oligonucleotide is 5-20 nucleotides in length.

7. The sensing element of claim 5, wherein
the oligonucleotide comprises at least 90% thymine, adenine, or a mixture of thymine and adenine.

8. The sensing element of claim 5, wherein
the oligonucleotide is selected from the group consisting of SEQ ID NO: 8, SEQ ID NO: 9, SEQ ID NO: 10, SEQ ID NO: 11, SEQ ID NO: 12 and SEQ ID NO: 13.

9. The sensing element of claim 1, wherein
the charge transfer-modulating moiety is functionalized with one or more redox reporters.

10. The sensing element of claim 6, wherein
the charge transfer-modulating moiety is not functionalized with a redox reporter.

11. The sensing element of claim 1, wherein
the redox reporter of the recognition element and/or charge transfer-modulating moiety is selected from the group consisting of methylene blue, ferrocene, viologen, anthraquinone or any other quinones, ethidium bromide, daunomycin, organo-metallic redox labels, for example porphyrin complexes or crown ether cycles or linear ethers, ruthenium, bis-pyridine, tris-pyridine, bis-imidizole, ethylenetetracetic acid-metal complexes, cytochrome c, plastocyanin, and cytochrome c′.

12. The sensing element of claim 1, wherein
the sensing element is configured for in vivo use.

13. A method of drift-corrected measurement of the concentration of a target species in a sample by the use of an electrochemical sensor, wherein the electrochemical sensor comprises a sensing element, wherein the sensing element comprises:
an electrode: wherein the electrode is functionalized with a plurality of a recognition elements, wherein the recognition elements are capable of selectively binding a target species and wherein the recognition elements are functionalized with one or more redox reporters; and
wherein the electrode is functionalized with a plurality of a charge transfer-modulating moieties, wherein the charge-transfer modulating moieties do not substantially bind to the target species:
the method comprising the steps of:
deploying the sensing element of the electrochemical sensor such that it is exposed to a sample;
applying a series of excitation pulses to the sensing element at a selected non-responsive frequency to generate a reference signal, wherein the non-responsive frequency is a frequency at which the signal not measurably affected by the presence of target, is minimally responsive to the presence of target, or is negatively responsive to the presence of target;
applying a series of excitation pulses to the electrochemical sensor at a selected responsive frequency to generate a measurement signal, wherein the responsive frequency is a frequency at which the signal is dependent upon the concentration of target in the sample;
subtracting the reference signal from the measurement signal to determine a drift-corrected signal value; and
applying a mathematical relationship between drift-corrected signal value and target concentration to the measured drift-corrected signal to determine the concentration of target in the sample.

14. The method of claim 13, wherein
the sample is unprocessed, the sample is whole blood, and/or the sensing element is deployed in vivo.

15-16. (canceled)

17. A method of measuring the concentration of a target species in a sample by use of an electrochemical sensor,
wherein the electrochemical sensor comprises a sensing element, wherein the sensing element comprises:
an electrode; wherein the electrode is functionalized with a plurality of a recognition elements, wherein the recognition elements are capable of selectively binding a target species and wherein the recognition elements are functionalized with one or more redox reporters; and
wherein the electrode is functionalized with a plurality of a charge transfer-modulating moieties, wherein the charge-transfer modulating moieties do not substantially bind to the target species:
the method comprising the steps of:
exposing the sensing element to a calibration sample of known target concentration and (1) obtaining a baseline reference signal by applying a series of excitation pulses to the sensing element at a selected non-responsive frequency, wherein the non-responsive frequency is a frequency at which the signal is not measurably affected by the presence of target, is minimally responsive to the presence of target, or is negatively responsive to the presence of target; and (2) obtaining a baseline measurement signal by applying a series of excitation pulses to the sensing element at a selected responsive frequency, wherein the responsive frequency is a frequency at which the signal is dependent upon the concentration of target in the sample;
exposing the sensing element to a sample of unknown target concentration and (1) obtaining a reference signal by applying a series of excitation pulses to the sensing element at the non-responsive frequency; and (2) obtaining a measurement signal by applying a series of excitation pulses to the sensing element at the responsive frequency;
calculating a normalized reference signal, which is the ratio of the reference signal obtained in the sample of unknown target concentration to the baseline reference signal measured in the sample of known target concentration;
calculating the normalized measurement signal, which is the ratio of the measurement signal obtained in the sample of unknown target concentration to the baseline measurement signal obtained in the sample of known target concentration;
subtracting the normalized reference signal, which is indicative of drift, from the normalized measurement signal to obtain a normalized drift-corrected signal; and
applying a mathematical relationship between normalized drift-corrected signal and target concentration to the measured value of normalized drift-corrected signal to determine the target concentration in the sample.

18. The method of claim 17, wherein
the sample is unprocessed, the sample is whole blood, and/or the sensing element is deployed in vivo.

19-20. (canceled)

21. A camptothecin binding aptamer, comprising
a polynucleotide selected from the group consisting of SEQ ID NO: 2, SEQ ID NO: 3, SEQ ID NO: 4, SEQ ID NO: 5, SEQ ID NO: 6 and SEQ ID NO: 7.
